(19) United States
(12) Patent Application Publication
(10) Pub. No.: US 2021/0171903 A1
(43) Pub. Date: Jun. 10, 2021

(54) UNIVERSAL DONOR STEM CELLS AND RELATED METHODS

(71) Applicant: President and Fellows of Harvard College, Cambridge, MA (US)

(72) Inventors: Torsten B. Meissner, Somerville, MA (US); Leonardo M.R. Ferreira, San Francisco, CA (US); Jack L. Strominger, Cambridge, MA (US); Chad A. Cowan, Boston, MA (US)

(21) Appl. No.: 16/908,618

(22) Filed: Jun. 22, 2020

Related U.S. Application Data
(63) Continuation of application No. 16/596,697, filed on Oct. 8, 2019, now abandoned.
Application No. 16/596,697 is a continuation of application No. 16/277,913, filed on Feb. 15, 2019, now abandoned.
(60) Provisional application No. 62/631,393, filed on Feb. 15, 2018.

(51) Int. Cl. C12N 5/0735; C07K 14/705; C07K 14/74

(52) U.S. Cl. CPC C12N 5/0606; C07K 14/70503; C07K 14/70539; C07K 14/70532

(57) ABSTRACT

Disclosed herein are universal donor stem cells and related methods of their use and production. The universal donor stem cells disclosed herein are useful for overcoming the immune rejection in cell-based transplantation therapies. In certain embodiments, the universal donor stem cells disclosed herein have modulated expression of one or more MHC-I and MHC-II human leukocyte antigens and one or more tolerogenic factors.

### CROSS-REFERENCE TO RELATED APPLICATIONS

This application is a continuation application of U.S. application Ser. No. 16/596,697, filed on Oct. 8, 2019, which is a continuation of U.S. application Ser. No. 16/277,913, filed on Feb. 15, 2019, which claims the benefit of U.S. Provisional Application No. 62/631,393, filed on Feb. 15, 2018. The entire teachings of the above applications are incorporated herein by reference.

### BACKGROUND OF THE INVENTION

Therapies utilizing human pluripotent stem cell-derived cells for transplantation have the potential to revolutionize the way diseases are treated. A major obstacle for their clinical translation is the rejection of allogeneic cells by the recipient's immune system. Strategies aiming at overcoming this immune barrier include banking cells with defined HLA haplotypes (Nakajima et al., 2007; Taylor et al., 2005) and the generation of patient-specific induced pluripotent stem cells (iPSCs) (Takahashi et al., 2007; Yu et al., 2007). However, multiple limitations (de Rham and Villard, 2014; Tapia and Scholer, 2016) prohibit the broader use of these approaches and emphasize the need for “off-the-shelf” cell products that can be readily administered to any patient in need.

### SUMMARY OF THE INVENTION

Disclosed herein are efficient strategies to overcome immune rejection in cell-based transplantation therapies by the creation of universal donor stem cell lines.

Disclosed herein are stem cells comprising modulated expression of one or more MHC-I and MHC-II human leukocyte antigens and one or more tolerogenic factors relative to a wild-type stem cell.

In some embodiments, the one or more MHC-I human leukocyte antigens are selected from the group consisting of HLA-A, HLA-B, and HLA-C. In some aspects, the modulated expression of the one or more MHC-I human leukocyte antigens comprises reduced expression of the one or more MHC-I human leukocyte antigens. In some embodiments, the one or more MHC-I human leukocyte antigens are deleted from the genome of the cell, thereby modulating the expression of the one or more MHC-I human leukocyte antigens.

In some embodiments, the one or more MHC-II human leukocyte antigens are selected from the group consisting of HLA-DP, HLA-DQ, and HLA-DR. In some aspects, the modulated expression of the one or more MHC-II human leukocyte antigens comprises reduced expression of the one or more MHC-II human leukocyte antigens. In some embodiments, one or more indels were introduced into CIITA, thereby modulating the expression of the one or more MHC-II human leukocyte antigens.

In some embodiments, the cell does not express HLA-A, HLA-B, and HLA-C. In certain aspects, the cell is an HLA-A^{-/-}, HLA-B^{-/-}, HLA-C^{-/-}, and CIITA^{indel/indel} cell.

In some embodiments, the one or more tolerogenic factors are selected from the group consisting of HLA-G, PD-L1, and CD47. In certain aspects, the modulated expression of the one or more tolerogenic factors comprises increased expression of the one or more tolerogenic factors. In some embodiments, the one or more tolerogenic factors are inserted into an AAVS1 safe harbor locus. In some aspects, HLA-G, PD-L1, and CD47 are inserted into an AAVS1 safe harbor locus. In some embodiments, the one or more tolerogenic factors inhibit immune rejection.

In some embodiments, the stem cell is an embryonic stem cell. In some aspects, the stem cell is a pluripotent stem cell. In some embodiments, the stem cell is hypoimmunogenic. In some aspects, the stem cell is a human stem cell.

In some embodiments, the stem cell retains pluripotency. In some aspects, the stem cell retains differentiation potential. In some embodiments, the stem cell exhibits reduced T cell response. In some aspects, the stem cell exhibits protection from NK cell response. In some embodiments, the stem cell exhibits reduced macrophage engulfment.

Also disclosed herein are stem cells that do not express HLA-A, HLA-B, HLA-C, HLA-DP, HLA-DQ, and HLA-DR.

In some embodiments, the stem cell is a HLA-A^{-/-}, HLA-B^{-/-}, HLA-C^{-/-}, and CIITA^{indel/indel} cell. In some aspects, the stem cell expresses tolerogenic factors HLA-G, PD-L1, and CD47. In some embodiments, the tolerogenic factors are inserted into an AAVS1 safe harbor locus. In certain aspects, the tolerogenic factors inhibit immune rejection.

In some embodiments, the stem cell is an embryonic stem cell. In some aspects, the stem cell is a pluripotent stem cell. In some embodiments, the stem cell is hypoimmunogenic.

Disclosed herein are methods of preparing a hypoimmunogenic stem cell, the method comprising modulating expression of one or more MHC-I and MHC-II human leukocyte antigens and one or more tolerogenic factors of a stem cell relative to a wild-type stem cell, thereby preparing the hypoimmunogenic stem cell.

In some embodiments, the one or more MHC-I human leukocyte antigens are selected from the group consisting of HLA-A, HLA-B, and HLA-C. In some aspects, the modulated expression of the one or more MHC-I human leukocyte antigens comprises reduced expression of the one or more MHC-I human leukocyte antigens. In some embodiments, the one or more MHC-I human leukocyte antigens are deleted from the genome of the stem cell, thereby modulating the expression of the one or more MHC-I human leukocyte antigens.

In some embodiments, the one or more MHC-II human leukocyte antigens are selected from the group consisting of HLA-DP, HLA-DQ, and HLA-DR. In some aspects, the modulated expression of the one or more MHC-II human leukocyte antigens comprises reduced expression of the one or more MHC-II human leukocyte antigens. In some embodiments, one or more indels were introduced into CIITA, thereby modulating the expression of the one or more MHC-II human leukocyte antigens.

In some aspects, the hypoimmunogenic stem cell does not express HLA-A, HLA-B, and HLA-C. In some embodiments, the hypoimmunogenic stem cell is an HLA-A^{-/-}, HLA-B^{-/-}, HLA-C^{-/-}, and CIITA^{indel/indel} cell.

In some embodiments, the one or more tolerogenic factors are selected from the group consisting of HLA-G, PD-L1, and CD47. In some aspects, the modulated expression of the one or more tolerogenic factors comprises increased expression of the one or more tolerogenic factors. In some embodiments, the one or more tolerogenic factors are inserted into an AAVS1 safe harbor locus. In some aspects, HLA-G, PD-L1, and CD47 are inserted into an AAVS1 safe harbor locus. In some embodiments, the one or more tolerogenic factors inhibit immune rejection.

In some embodiments, the hypoimmunogenic stem cell retains pluripotency. In some aspects, the hypoimmunogenic stem cell retains differentiation potential. In some embodiments, the hypoimmunogenic stem cell exhibits reduced T cell response. In some aspects, the hypoimmunogenic stem cell exhibits protection from NK cell response. In some embodiments, the hypoimmunogenic stem cell exhibits reduced macrophage engulfment.

In some embodiments, the stem cell is contacted with a Cas protein or a nucleic acid sequence encoding the Cas protein and a first pair of ribonucleic acids having sequences SEQ ID NOS: 1-2, thereby editing the HLA-A gene to reduce or eliminate HLA-A surface expression and/or activity in the stem cell. In some aspects, the stem cell is contacted with a Cas protein or a nucleic acid sequence encoding the Cas protein and a first pair of ribonucleic acids having sequences SEQ ID NOS: 3-4, thereby editing the HLA-B gene to reduce or eliminate HLA-B surface expression and/or activity in the stem cell. In some aspects, the stem cell is contacted with a Cas protein or a nucleic acid sequence encoding the Cas protein and a first pair of ribonucleic acids having sequences SEQ ID NOS: 5-6, thereby editing the HLA-C gene to reduce or eliminate HLA-C surface expression and/or activity in the stem cell. In some aspects, the stem cell is contacted with a Cas protein or a nucleic acid sequence encoding the Cas protein and a ribonucleic acid having sequence SEQ ID NO: 7, thereby introducing indels into CIITA to reduce or eliminate MHC-II human leukocyte antigens surface expression and/or activity in the stem cell.

Also disclosed herein are methods of preparing a hypoimmunogenic stem cell, the method comprising modulating expression of one or more MHC-I and MHC-II human leukocyte antigens and one or more tolerogenic factors of a stem cell relative to a wild-type stem cell, thereby preparing the hypoimmunogenic stem cell, wherein the stem cell is contacted with a Cas protein or a nucleic acid sequence encoding the Cas protein and a first pair of ribonucleic acids having sequences SEQ ID NOS: 1-2, thereby editing the HLA-A gene to reduce or eliminate HLA-A surface expression and/or activity in the stem cell, wherein the stem cell is contacted with a Cas protein or a nucleic acid sequence encoding the Cas protein and a second pair of ribonucleic acids having sequences SEQ ID NOS: 3-4, thereby editing the HLA-B gene to reduce or eliminate HLA-B surface expression and/or activity in the stem cell, wherein the stem cell is contacted with a Cas protein or a nucleic acid sequence encoding the Cas protein and a third pair of ribonucleic acids having sequences SEQ ID NOS: 5-6, thereby editing the HLA-C gene to reduce or eliminate HLA-C surface expression and/or activity in the stem cell, and wherein the stem cell is contacted with a Cas protein or a nucleic acid sequence encoding the Cas protein and a ribonucleic acid having sequence SEQ ID NO: 7, thereby introducing indels into CIITA to reduce or eliminate MHC-II human leukocyte antigens surface expression and/or activity in the stem cell.

Also disclosed herein are methods of transplanting at least one hypoimmunogenic stem cell into a patient, wherein the hypoimmunogenic stem cell comprises modulated expression of one or more MHC-I and MHC-II human leukocyte antigens and one or more tolerogenic factors relative to a wild-type stem cell.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1J demonstrate genome editing ablates polymorphic HLA-A/-B/-C and HLA class II expression and enables expression of immunomodulatory factors from AAVS1 safe harbor locus. FIG. 1A provides a schematic representation of HLA-B and HLA-C CRISPR/Cas9 knockout strategy. Each pair of scissors represents two sgRNAs. Purple, red, and green arrows indicate primers used for PCR screening. FIG. 1B provides a schematic representation of HLA-A knockout strategy. Each pair of scissors represents one sgRNA. Yellow arrows show primers used for PCR screening. FIG. 1C provides FACS contour plots demonstrating successful ablation of HLA-A/B/C in HUES8. Wild-type (WT) or HLA-A/B/C knockout (KO) cells were treated with IFNγ for 48 hrs before staining with the indicated antibodies. FIG. 1D shows targeting strategy of CIITA locus. Blue arrows indicate primers used for PCR and Sanger sequencing. FIG. 1E shows HLA-DR mean fluorescence intensity (MFI) in differentiated CD144⁺ WT and KO ECs. FIG. 1F provides a schematic describing the genotypes of WT, KO, KI-PHC, and KIPC cell lines. FIG. 1G shows knock-in strategy of immune modulatory molecules. Scissors represent the sgRNA targeting the AAVS1 locus. Black and gray arrows indicate primers used for PCR screening. FIG. 1H shows PD-L1 and HLA-G expression in KI-PHC cells. FIG. 1I shows CD47 expression in KI-PHC cells. MFIs relative to WT cells are indicated on the right of histograms. FIG. 1J shows PD-L1 and CD47 expression in KI-PC cells.

FIGS. 2A-2E demonstrate KO and KI cell lines retain pluripotency and differentiation potential. FIG. 2A shows immunofluorescence indicating that pluripotency markers were expressed by WT, KO, KI-PHC, and KI-PC human pluripotent stem cells (hPSCs). Scale bars, 200 μm. FIG. 2B shows qRT-PCR was carried out to survey trilineage markers after WT, KO, KI-PHC, and KI-PC hPSCs were differentiated into the indicated three germ layers. Relative quantification was normalized to each gene level in unmodified hPSCs. FIG. 2C shows G-banding of chromosomes in KO, KI-PHC, and KI-PC cell lines demonstrated normal karyotypes after successive rounds of genome engineering. FIG. 2D provides a table showing the PCR-based analyses of exonic off-target sites in engineered hPSC lines. FIG. 2E shows target capture sequencing results showing the % reads with altered sequence at off-target sites in WT and engineered hPSC lines. Black circle, SNP/polymorphism (PM) site; red circle, edited off-target site; blue circle, CIITA on-target site as positive control.

FIGS. 3A-3D demonstrate reduced T cell activities against KO and KI-PHC cell lines in vitro. FIG. 3A provides scatterplots displaying the percent of proliferating T cells in CD3⁺ (left panel, n=8 donors), CD4⁺ (middle panel, n=6 donors), and CD8⁺ T cell populations (right panel, n=6 donors) when co-cultured for 5 days with WT, KO, or KI ECs. T cells cultured alone were used as negative control; T cells activated with CD3/CD28 beads served as positive controls. Paired one-way ANOVA followed by Tukey's multiple comparison test. Data are mean±s.e.m.; *p<0.05; **p<0.01. FIG. 3B provides a scatterplot displaying the percentage of CD69⁺ (upper panel) and CD25⁺ cells (lower panel) in CD3⁺ (left panel), CD4⁺ (middle panel), and CD8⁺ T cell populations (right panel) after a five-day co-culture with WT, KO, or KI ECs (n=11 donors in all plots). The same negative and positive controls were used as in A. Paired one-way ANOVA followed by Tukey's multiple comparison test. Data are mean±s.e.m.; **p<0.01; ***p<0.001; ****p<0.0001. FIG. 3C provides bar graphs of IFNγ (left panel) and IL-10 (right panel) concentration in the medium following co-culture of WT, KO, or KI ECs with CD3⁺ T cells from one representative donor. Spontaneous release from T cells alone were used as negative controls. Ordinary one-way ANOVA followed by Tukey's multiple comparison test. Data are mean±s.d.; **p<0.01; ***p<0.001. FIG. 3D provides a bar graph representing percent T cell cytotoxicity against WT, KO, and KI ECs (n=6 donors). LDH release assay was performed and the percentage of T cell cytotoxicity from each donor was calculated. Paired one-way ANOVA followed by Tukey's multiple comparison test. Data are mean±s.e.m.; *p<0.05; **p<0.01.

FIGS. 4A-4E demonstrate reduced T cell responses against KO and KI cell lines in vivo. FIG. 4A provides a schematic describing the pre-sensitization of allogeneic CD8⁺ T cells and the workflow of in vivo T cell recall response assay. FIG. 4B shows percentage of increased teratoma volume on day 5 or 7 post T cell injection compared to day 0. Genotype of teratoma: WT (n=9), KO (n=7), KI-PHC (n=6), and KI-PC (n=7). Ordinary one-way ANOVA followed by Tukey's multiple comparison test. Data are mean±s.e.m.; *p<0.05. FIG. 4C shows percentage of increased teratoma volume on day 0 of T cell injection compared to 2 days pre-injection. Genotype of teratoma: WT (n=9), KO (n=7), KI-PHC (n=6), and KI-PC (n=7). FIG. 4D shows relative hCD8 (left panel) and IL-2 (right panel) mRNA expression in WT (n=8), KO (n=7), KI-PHC (n=6), and KI-PC (n=7) teratomas harvested on day 8 post-T cell injection. The expression was normalized to RPLP0. Ordinary one-way ANOVA followed by Tukey's multiple comparison test. Data are mean±s.e.m.; *p<0.05; **p<0.01. FIG. 4E shows representative hematoxylin and eosin (H&E) staining of WT, KO, KI-PHC, and KI-PC teratomas harvested on day 8 post T cell injection. The black arrows indicate the sites of T cell infiltration. Scale bars, 100 μm.

FIGS. 5A-5D demonstrate KI cell lines are protected from NK cell and macrophage responses. FIG. 5A provides a scatterplot of NK cell degranulation against WT, KO, or KI-PHC VSMCs (n=7 donors). The percentage of degranulating NK cells was plotted as % CD107a-expressing CD56⁺ cells for each donor. NK cells cultured alone were used as negative control; NK cells treated with PMA/ionomycin served as positive control. Paired one-way ANOVA followed by Tukey's multiple comparison test. Data are mean±s.e.m.; **p<0.01. FIG. 5B provides a bar graph representing the percentage of NK cytotoxicity against WT, KO, and KI-PHC VSMCs from one representative donor at the indicated effector/target (E/T) ratios (n=3 replicates). LDH release assay was performed and the % NK cytotoxicity was calculated as specific lysis of NK cell-killed VSMCs relative to maximum cell lysis. Unpaired one-way ANOVA followed by Tukey's multiple comparison test. Data are mean±s.d.; *p<0.05; ***p<0.001. FIG. 5C provides time-lapse plots of macrophage phagocytosis assay (n=5 monocyte donors). pHrodo-red-labelled VSMCs of indicated genotypes that were pretreated (right panel) or not pretreated (left panel) with Staurosporine (STS) were co-incubated with monocyte-derived macrophages for 6 hrs Images were acquired every 20 min using Celldiscover 7 live cell imaging system. Total integrated fluorescence intensity of pHrodored+phagosomes per image was analyzed. Data are mean±s.e.m. FIG. 5D provides scatterplots of macrophage phagocytosis assay at 4 hr co-incubation (n=9 monocyte donors, three independent experiments). The experimental conditions were the same as in FIG. 5C. Paired one-way ANOVA followed by Tukey's multiple comparison test. Data are mean±s.e.m.; *p<0.05; **p<0.01. VSMC=vascular smooth muscle cells; NK=natural killer cells.

FIGS. 6A-6G demonstrate genome editing ablates polymorphic HLA-A/-B/-C and HLA class II expression and enables expression of immunomodulatory factors from AAVS1 safe harbor locus. FIG. 6A shows PCR confirmation of HLA-B/-C knockout using primers shown in FIG. 1A. FIG. 6B shows PCR confirmation of HLA-A knockout using primers shown in FIG. 1B. FIG. 6C shows PCR products using the primers flanking the CIITA cutting site. FIG. 6D shows Sanger sequencing reveals that in the KO cell line, 1 bp (shown in red) was inserted on one CIITA allele and 12 bp (shown as dashes) were deleted from the other allele. FIG. 6E shows CD144 expression in differentiated WT and KO endothelial cells (ECs). FIG. 6F shows workflow of generating KO and KI ES cell lines. FIG. 6G shows PCR confirmation of knock-in of the KI-PHC/KI-PC constructs using primers shown in FIG. 1G.

FIGS. 7A-7H demonstrate genome editing ablates polymorphic HLA-A/-B/-C and HLA class II expression and enables expression of immunomodulatory factors from AAVS1 safe harbor locus. FIG. 7A shows CD47 expression in WT and KI-PC ES cells. MFIs relative to WT cells are given on the right of the histograms. FIG. 7B shows HLA-A2 expression in WT, KI-PHC, and KI-PC ES cells post IFNγ treatment confirming the ablation of classical HLA class Ia molecules in the KI cell lines. FIG. 7C shows CD144 expression in differentiated WT, KI-PHC, and KI-PC ECs. FIG. 7D shows HLA-DR mean fluorescence intensity (MFI) confirming the ablation of HLA class II in differentiated KI-PHC and KI-PC ECs. HLA-DR expression was analyzed on CD144⁺ cells. FIG. 7E shows CD140b expression in differentiated WT, KO, KI-PHC, and KI-PC VSMCs. FIG. 7F provides contour plots showing the expression of PD-L1 and HLA-G in differentiated WT and KI-PHC VSMCs (upper left panel). CD47 expression in differentiated WT and KI-PHC VSMCs (upper right panel). Contour plots showing the expression of PD-L1 and CD47 in differentiated WT and KI-PC VSMCs (lower left panel). CD47 expression in differentiated WT and KI-PC VSMCs (lower right panel). FIG. 7G shows HLA-E expression in differentiated WT, B2M^{-/-}, KO, and KI-PHC VSMCs upon IFN-γ stimulation. Gray, isotype; colored, antibodies. FIG. 7H shows relative HLA-E mRNA expression in differentiated WT, B2M^{-/-}, KO, and KI-PHC VSMCs with or without IFN-γ stimulation.

FIG. 8 provides sequencing chromatograms of predicted exonic off-target sites in gene-modified hPSC lines and in parental WT cells.

FIG. 9 shows Sequence inspection from NGS showing editing at off-target sites in engineered hPSC lines, and the SNP/polymorphic sites observed in engineered lines as well as WT cells.

FIGS. 10A-10D demonstrate reduced T cell activities against KO and KI-PHC cell lines. FIG. 10A shows gating strategy used in T cell proliferation and activation assays. FIG. 10B provides a T cell proliferation assay of one representative donor using WT, KO, and KI-PHC ECs as target cells. CD3⁺ (top panel), CD4⁺ (middle panel), and CD8⁺ (bottom panel). T cells cultured alone were used as negative control; T cells treated with CD3/CD28 beads served as positive control. FIG. 10C shows doxycycline-inducible PD-L1 expression in WT VSMCs. FIG. 10D provides a scatterplot of percent proliferating T cells in CD3⁺ (left panel), CD4⁺ (middle panel), and CD8⁺ T cell populations (right panel) co-cultured for 7 days with VSMCs in the presence or absence of doxycycline-induced PD-L1 expression (n=4 donors). T cells with reduced CFSE signal were quantified as proliferating cells. T cells cultured alone served as negative control; T cells activated with CD3/CD28 beads were used as positive control. Paired two tailed t-test; Data are mean±s.e.m.; *p<0.05; ns, no significance.

FIGS. 11A-11E demonstrate KI cell lines are protected from NK cell and macrophage responses. FIG. 11A shows CD69 and PD-1 expression examined in pre and post priming of one representative CD8⁺ T donor. FIG. 11B provides gating strategy of NK cell degranulation assay. FIG. 11 C provides FACS contour plots of NK cell degranulation assay for one representative donor. FIG. 11D shows CD47 MFI confirming the ablation of CD47 expression in differentiated CD47^{-/-} VSMCs. FIG. 11E provides fluorescence images showing engulfed VSMCs pre-labeled with pHrodo-Red after 4 h co-incubation with macrophages from one representative donor. VSMCs were either pretreated with staurosporine or left untreated. The images represent overlays of bright field and red channel and the fluorescent phagosomes are highlighted after masking by the ZEN imaging analysis software. Scale bars, 200 μm.

FIGS. 12A-12C demonstrate overcoming the HLA barrier. FIG. 12A provides a schematic representation of the MHC class II and class I enhanceosomes. Targeting of CIITA, the master regulator of MHC class II expression, prevents MHC class II expression. The promoters of MHC class I genes are more complex, and thus deletion of NLRC5, a CIITA homologues regulating MHC class I expression, results in only a reduction of MHC class I expression. FIG. 12B shows reduction of IFNg-induced MHC class I expression in NLRC5−/−CIITA−/hPSCs. WT, or the indicated KO HUES9 cells were stimulated with IFNg for 48 hrs and subsequently stained for MHC class I expression, recorded by FACS. Deletion of the accessory chain B2M prevents MHC class I surface expression entirely, but will render these cells susceptible to NK cell killing. FIG. 12C shows targeting strategy to selectively remove the polymorphic HLA genes HLA-A/B/C from the genome of hPSCs. Schematic representations of targeting strategy are provided. Also shown is PCR confirmation of the respective deletions in the genome of HUES8.

FIGS. 13A-13E demonstrate knock-in (KI) strategy of tolerogenic factors into a safe harbor locus. FIGS. 13A-13B provide schematic representation of the KI constructs. FIG. 13C shows confirmation of the loss of HLA class I expression in two KI clones (C8 and C12). FIG. 13D shows successful over expression of PD-L1 and CD47 in the HLA deficient KI clones C8 and C12 from the AAVS1 safe harbor locus. FIG. 13E shows the ultimate goal is to reverse engineer the immunomodulatory activity of human trophoblasts (PD-L1, HLA-G, CD47 high) which induce tolerance to a semiallogeneic fetus (50% of paternal and thus foreign origin) during pregnancy.

FIGS. 14A-14B demonstrate functional immune-silent cells for transplantation. FIG. 14A shows confirmation of HLA expression in modified hPSC (HUES8). Loss of MHC class I expression was confirmed in two independent HLA-A/B/C−/−CIITA−/− KO clones—D1 and F2—by FACS. Similar morphology of KO clone-derived endothelial cells (EC) was seen. IFNγ-induced MHC class II expression in EC of the indicated genotypes, demonstrates loss of HLA class II in the HLA-A/B/C−/−CIITA−/− KO clones. FIG. 14B shows a T cell proliferation assay (top panel) and a NK cell degranulation assay (bottom panel). For the T cell proliferation assay (top panel), a CFSE-labelled T cell clone was used to assess T cell proliferation against EC derived from HUES9 of the indicated genotypes. Loss of CFSE signal is proportional to T cell proliferation, a proxy of the immunostimulatory activity of those cells. While WT EC trigger prominent T cell proliferation over a 7 day period, T cell proliferation is reduced in the presence of two independent NLRC5−/−CIITA−/− KO clones and absent when co-incubated with B2M−/−CIITA−/− KO EC. For the NK cell degranulation assay (bottom panel), an HLA-deficient VSMC (D1, F2) trigger enhanced NK cell degranulation when compared to WT cells. PMA/Ionomycin or HLA-deficient 221 cells were used as positive control for NK degranulation. NC=negative control, NK cells only.

FIGS. 15A-15B demonstrate generation of preclinical data. FIG. 15A shows improved engraftment of immune silent human pluripotent stem cells in humanized mice. NSG mice reconstituted with a human immune system (BLT), were transplanted with ES cells of the indicated genotype, and allowed to form teratoma. 4-6 weeks post transplantation teratoma size and consistency were scored in a blinded manner. While the WT teratoma show hallmarks of rejection, growth of the NLCR5−/−CIITA−/− and B2M−/−CIITA−/− stem cells was found less restricted, suggesting they are immune-protected. FIG. 15B shows introduction of an inducible Caspase9 (iCasp9) killing switch can ablate cells upon treatment with the CID dimerizer. Cartoon of the iCasp9 killing switch (left). Dose titration of the CID dimerizer and time course in transiently transfected 293T cells (right). Ultimately, the iCasp9 killing switch will be integrated into a safe harbor locus of the modified, immune silent stem cells.

### DETAILED DESCRIPTION OF THE INVENTION

The inventions disclosed herein employ genome editing technologies (e.g., the CRISPR/Cas or TALEN systems) to reduce or eliminate expression of critical immune genes or, in certain instances, insert tolerance-inducing factors, in stem cells, rendering them and the differentiated cells prepared therefrom hypoimmunogenic and less prone to immune rejection by a subject into which such cells are transplanted.

As used herein to characterize a cell, the term “hypoimmunogenic” generally means that such cell is less prone to immune rejection by a subject into which such cells are transplanted. For example, relative to an unaltered wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. In some aspects, genome editing technologies (e.g., the CRISPR/Cas or TALEN systems) are used to modulate (e.g., reduce or eliminate) the expression of MHC-I and MHC-II genes.

In certain embodiments, the inventions disclosed herein relate to a stem cell, the genome of which has been altered to reduce or delete critical components of HLA expression. Similarly, in certain embodiments, the inventions disclosed herein relate to a stem cell, the genome of which has been altered to insert one or more tolerance inducing factors. The present invention contemplates altering target polynucleotide sequences in any manner which is available to the skilled artisan, for example, utilizing a TALEN, ZFN, or a CRISPR/Cas system. Such CRISPR/Cas systems can employ a variety of Cas proteins (Haft et al. PLoS Comput Biol. 2005; 1(6)e60). In some embodiments, the CRISPR/Cas system is a CRISPR type I system. In some embodiments, the CRISPR/Cas system is a CRISPR type II system. In some embodiments, the CRISPR/Cas system is a CRISPR type V system. It should be understood that although examples of methods utilizing CRISPR/Cas (e.g., Cas9 and Cpf1) and TALEN are described in detail herein, the invention is not limited to the use of these methods/systems. Other methods of targeting polynucleotide sequences to reduce or ablate expression in target cells known to the skilled artisan can be utilized herein.

The present inventions contemplate altering, e.g., modifying or cleaving, target polynucleotide sequences in a cell for any purpose, but particularly such that the expression or activity of the encoded product is reduced or eliminated. In some embodiments, the target polynucleotide sequence in a cell (e.g., ES cells or iPSCs) is altered to produce a mutant cell. As used herein, a “mutant cell” generally refers to a cell with a resulting genotype that differs from its original genotype or the wild-type cell. In some instances, a “mutant cell” exhibits a mutant phenotype, for example when a normally functioning stem gene is altered using the CRISPR/Cas systems. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system can be used to induce an indel of any length or a point mutation in a target polynucleotide sequence.

In some embodiments, the alteration results in a knock out of the target polynucleotide sequence or a portion thereof. For example, knocking out a target polynucleotide sequence in a cell can be performed in vitro, in vivo or ex vivo for both therapeutic and research purposes. Knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

As used herein, “knock out” includes deleting all or a portion of the target polynucleotide sequence in a way that interferes with the function of the target polynucleotide sequence or its expression product.

In some embodiments, the alteration results in reduced expression of the target polynucleotide sequence. The terms “decrease,” “reduced,” “reduction,” and “decrease” are all used herein generally to mean a decrease by a statistically significant amount. However, for avoidance of doubt, “decreased,” “reduced,” “reduction,” “decrease” includes a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

In some embodiments, the alteration is a homozygous alteration. In some embodiments, the alteration is a heterozygous alteration.

In some embodiments, the alteration results in correction of the target polynucleotide sequence from an undesired sequence to a desired sequence. CRISPR/Cas systems can be used to correct any type of mutation or error in a target polynucleotide sequence. For example, CRISPR/Cas systems can be used to insert a nucleotide sequence that is missing from a target polynucleotide sequence due to a deletion. CRISPR/Cas systems can also be used to delete or excise a nucleotide sequence from a target polynucleotide sequence due to an insertion mutation. In some instances, CRISPR/Cas systems can be used to replace an incorrect nucleotide sequence with a correct nucleotide sequence (e.g., to restore function to a target polynucleotide sequence that is impaired due to a loss of function mutation).

CRISPR/Cas systems can alter target polynucleotides with surprisingly high efficiency. In certain embodiments, the efficiency of alteration is at least about 5%. In certain embodiments, the efficiency of alteration is at least about 10%. In certain embodiments, the efficiency of alteration is from about 10% to about 80%. In certain embodiments, the efficiency of alteration is from about 30% to about 80%. In certain embodiments, the efficiency of alteration is from about 50% to about 80%. In some embodiments, the efficiency of alteration is greater than or equal to about 80%. In some embodiments, the efficiency of alteration is greater than or equal to about 85%. In some embodiments, the efficiency of alteration is greater than or equal to about 90%, about 91%, about 92%, about 93%, about 94%, about 95%, about 96%, about 97%, about 98%, or about 99%. In some embodiments, the efficiency of alteration is equal to about 100%.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, CRISPR/Cas systems include a Cas protein or a nucleic acid sequence encoding the Cas protein and at least one to two ribonucleic acids (e.g., gRNAs) that are capable of directing the Cas protein to and hybridizing to a target motif of a target polynucleotide sequence. In some embodiments, CRISPR/Cas systems include a Cas protein or a nucleic acid sequence encoding the Cas protein and a single ribonucleic acid or at least one pair of ribonucleic acids (e.g., gRNAs) that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosolated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprise a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to Cast, Cas2, Cas3, Cas4, Cas5, Cas6, Cas7, Cas8 and Cas9. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5 h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6.

In some embodiments, the Cas protein is Cas9 protein or a functional portion thereof. In some embodiments, the Cas protein is Cas9 from any bacterial species or functional portion thereof. Cas9 protein is a member of the type II CRISPR systems which typically include a trans-coded small RNA (tracrRNA), endogenous ribonuclease 3 (mc) and a Cas protein. Cas 9 protein (also known as CRISPR-associated endonuclease Cas9/Csn1) is a polypeptide comprising 1368 amino acids. Cas 9 contains 2 endonuclease domains, including an RuvC-like domain (residues 7-22, 759-766 and 982-989) which cleaves target DNA that is non-complementary to crRNA, and an HNH nuclease domain (residues 810-872) which cleave target DNA complementary to crRNA.

In some embodiments, the Cas protein is Cpf1 protein or a functional portion thereof. In some embodiments, the Cas protein is Cpf1 from any bacterial species or functional portion thereof. Cpf1 protein is a member of the type V CRISPR systems. Cpf1 protein is a polypeptide comprising about 1300 amino acids. Cpf1 contains a RuvC-like endonuclease domain. Cpf1 cleaves target DNA in a staggered pattern using a single ribonuclease domain. The staggered DNA double-stranded break results in a 4 or 5-nt 5′ overhang.

As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cpf1 protein functional domains selected from the group consisting of a DNA binding domain, at least one RNA binding domain, a helicase domain, and an endonuclease domain. In some embodiments, the functional domains form a complex.

It should be appreciated that the present invention contemplates various ways of contacting a target polynucleotide sequence with a Cas protein (e.g., Cas9). In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas protein comprises a Cas polypeptide fused to a cell-penetrating peptide. In some embodiments, the Cas protein comprises a Cas polypeptide fused to a PTD.

In some embodiments, the Cas protein can be introduced into a cell containing the target polynucleotide sequence in the form of a nucleic acid encoding the Cas protein (e.g., Cas9 or Cpf1). The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, nucleic acids encoding Cas protein and nucleic acids encoding the at least one to two ribonucleic acids are introduced into a cell via viral transduction (e.g., lentiviral transduction).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods of the present invention contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids of the present invention can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, at least one of the one to two ribonucleic acids comprises a sequence selected from the group consisting of the ribonucleic acid sequences of SEQ ID NOs: 1-7. In some embodiments, at least one ribonucleic acid comprises a sequence selected from the group consisting of the ribonucleic acid sequences of SEQ ID NOs: 1-7.

In some embodiments, at least one of the one to two ribonucleic acids comprises a sequence with a single nucleotide mismatch to a sequence selected from the group consisting of the ribonucleic acid sequences of SEQ ID NOs: 1-7. In some embodiments, at least one ribonucleic acid comprises a sequence with a single nucleotide mismatch to a sequence selected from the group consisting of the ribonucleic acid sequences of SEQ ID NOs: 1-7.

In some embodiments, each of the one to two ribonucleic acids comprises guide RNAs that directs the Cas protein to and hybridizes to a target motif of the target polynucleotide sequence in a cell. In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, the target motif is a 17 to 23 nucleotide DNA sequence. In some embodiments, the target motif is at least 20 nucleotides in length. In some embodiments, the target motif is a 20-nucleotide DNA sequence.

In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. Those skilled in the art will appreciate that a variety of techniques can be used to select suitable target motifs for minimizing off-target effects (e.g., bioinformatics analyses). In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some aspects, the target polynucleotide sequence in a cell is altered to reduce or eliminate expression and/or activity of one or more critical immune genes in the cell using a genetic editing system (e.g., TALENs, ZFN, CRISPR/Cas, etc.). In some embodiments, the present disclosure provides that the target polynucleotide sequence in a cell is altered to delete a contiguous stretch of genomic DNA (e.g., delete one or more critical immune genes) from one or both alleles of the cell (e.g., using a CRISPR/Cas system). In some embodiments, the target polynucleotide sequence in a cell is altered to insert a genetic mutation in one or both alleles of the cell (e.g., using a CRISPR/Cas system). In still other embodiments, the universal stem cells disclosed herein may be subject to complementary genome editing approaches (e.g., using a CRISPR/Cas system), whereby such stem cells are modified to both delete contiguous stretches of genomic DNA (e.g., critical immune genes) from one or both alleles of the cell, as well as to insert one or more tolerance-inducing factors, such as HLA-G, CD47, and/or PD-L1, into one or both alleles of the cells to locally suppress the immune system and improve transplant engraftment.

The universal stem cells disclosed herein may be used, for example, to diagnose, monitor, treat and/or cure the presence or progression of a disease or condition in a subject (e.g., type 1 diabetes or multiple sclerosis). As used herein, a “subject” means a human or animal. In certain embodiments, the subject is a human. In certain embodiments, the subject is an adolescent. In certain embodiments, the subject is treated in vivo, in vitro and/or in utero. In certain aspects, a subject in need of treatment in accordance with the methods disclosed herein has a condition or is suspected or at increased risk of developing such condition. In some aspects, the universal stem cells are transplanted into a subject.

Provided herein are novel cells, compositions and methods that are useful for addressing such HLA-based immune rejection of transplanted cells.

### Ablation of MHC Class I and MHC Class II Genes

In certain aspects, the inventions disclosed herein relate to genomic modifications of one or more targeted polynucleotide sequences of the stem cell genome that regulates the expression of MHC-I and/or MHC-II human leukocyte antigens. In some aspects, a genetic editing system is used to modify one or more targeted polynucleotide sequences. In some aspects, a CRISPR/Cas system is used to delete the one or more targeted polynucleotide sequences and/or introduce indels into the one or more targeted polynucleotide sequences.

The efficient removal of the HLA barrier can be accomplished by targeting the polymorphic HLA alleles (HLA-A, -B, -C) directly and/or deletion of components of the MHC enhanceosomes, such as CIITA, that are critical for HLA expression.

In certain embodiments, HLA expression is interfered with. In some aspects, HLA expression is interfered with by targeting individual HLAs (e.g., knocking out expression of HLA-A, HLA-B and/or HLA-C) and/or targeting transcriptional regulators of HLA expression (e.g., CIITA). In some aspects multiple HLAs may be targeted at the same time. For example, HLA-B and HLA-C are adjacent and may be targeted simultaneously. In some aspects a 95 kb deletion of a stem cells genome using CRISPR/Cas may knock out HLA-B and HLA-C, as well as the promoters of the two genes. In some aspects a 13 kb deletion of a stem cells genome using CRISPR/Cas knocks out HLA-A, as well as the promoter of the gene.

In certain aspects, the stem cells disclosed herein do not express one or more human leukocyte antigens (e.g., HLA-A, HLA-B, HLA-C, HLA-DP, HLA-DQ and/or HLA-DR) corresponding to MHC-I and/or MHC-II and are thus characterized as being hypoimmunogenic. For example, in certain aspects, the stem cells disclosed herein have been modified such that the stem cell or a differentiated stem cell prepared therefrom does not express or exhibits reduced expression of one or more of the following MHC-I molecules: HLA-A, HLA-B and HLA-C. In some aspects, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene. In some aspects, the stem cells disclosed herein have been modified such that the stem cell or a differentiated stem cell prepared therefrom does not express or exhibits reduced expression of one or more of the following MHC-II molecules: HLA-DP, HLA-DQ, and HLA-DR. In some aspects, one or more indels are inserted into a transcriptional regulator of HLA class II expression (e.g., CIITA). A cell that has indels inserted into CIITA (e.g., targeting exon 1) may exhibit reduced or eliminated expression of HLA-DP, HLA-DQ, and/or HLA-DR.

In some aspects, the present disclosure provides a stem cell (e.g., hypoimmunogenic stem cell) or population thereof comprising a genome in which the HLA-A gene has been edited to delete a contiguous stretch of genomic DNA, thereby reducing or eliminating surface expression of MHC class I molecules in the cell or population thereof. The contiguous stretch of genomic DNA can be deleted by contacting the cell or population thereof with a Cas protein or a nucleic acid encoding the Cas protein and at least one ribonucleic acid or at least one pair of ribonucleic acids selected from the group consisting of SEQ ID NOs: 1-2.

In certain aspects, the present disclosure provides a method for altering a target HLA-A sequence in a cell comprising contacting the HLA-A sequence with a clustered regularly interspaced short palindromic repeats-associated (Cas) protein and at least one ribonucleic acid or at least one pair of ribonucleic acids, wherein the ribonucleic acids direct Cas protein to and hybridize to a target motif of the target HLA-A polynucleotide sequence, wherein the target HLA-A polynucleotide sequence is cleaved, and wherein the at least one ribonucleic acid or the at least one pair of ribonucleic acids is selected from the group consisting of SEQ ID NOs: 1-2.

In some aspects, the present disclosure provides a stem cell (e.g., hypoimmunogenic stem cell) or population thereof comprising a genome in which the HLA-B gene has been edited to delete a contiguous stretch of genomic DNA, thereby reducing or eliminating surface expression of MHC class I molecules in the cell or population thereof. The contiguous stretch of genomic DNA can be deleted by contacting the cell or population thereof with a Cas protein or a nucleic acid encoding the Cas protein and at least one ribonucleic acid or at least one pair of ribonucleic acids selected from the group consisting of SEQ ID NOs: 3-4.

In certain aspects, the present disclosure provides a method for altering a target HLA-B sequence in a cell comprising contacting the HLA-B sequence with a clustered regularly interspaced short palindromic repeats-associated (Cas) protein and at least one ribonucleic acid or at least one pair of ribonucleic acids, wherein the ribonucleic acids direct Cas protein to and hybridize to a target motif of the target HLA-B polynucleotide sequence, wherein the target HLA-B polynucleotide sequence is cleaved, and wherein the at least one ribonucleic acid or the at least one pair of ribonucleic acids is selected from the group consisting of SEQ ID NOs: 3-4.

In some aspects, the present disclosure provides a stem cell (e.g., hypoimmunogenic stem cell) or population thereof comprising a genome in which the HLA-C gene has been edited to delete a contiguous stretch of genomic DNA, thereby reducing or eliminating surface expression of MHC class I molecules in the cell or population thereof. The contiguous stretch of genomic DNA can be deleted by contacting the cell or population thereof with a Cas protein or a nucleic acid encoding the Cas protein and at least one ribonucleic acid or at least one pair of ribonucleic acids selected from the group consisting of SEQ ID NOs: 5-6.

In certain aspects, the present disclosure provides a method for altering a target HLA-C sequence in a cell comprising contacting the HLA-C sequence with a clustered regularly interspaced short palindromic repeats-associated (Cas) protein and at least one ribonucleic acid or at least one pair of ribonucleic acids, wherein the ribonucleic acids direct Cas protein to and hybridize to a target motif of the target HLA-C polynucleotide sequence, wherein the target HLA-C polynucleotide sequence is cleaved, and wherein the at least one ribonucleic acid or the at least one pair of ribonucleic acids is selected from the group consisting of SEQ ID NOs: 5-6.

In certain aspects, the present disclosure provides a stem cell (e.g., hypoimmunogenic stem cell) or population thereof comprising a genome in which the Class II transactivator (CIITA) gene has been edited to introduce one or more indels into exon 1, thereby reducing or eliminating surface expression of MHC class II molecules (e.g., HLA-DP, HLA-DQ, and HLA-DR) in the cell or population thereof. The one or more indels can be introduced by contacting the cell or population thereof with a Cas protein or a nucleic acid encoding the Cas protein and a ribonucleic acid consisting of SEQ ID NO: 7. In some aspects exon 1 of CIITA is targeted with the ribonucleic acid consisting of SEQ ID NO: 7 and at least one ribonucleic acid or at least one pair of ribonucleic acids selected from the group consisting of SEQ ID NOs: 1-2.

In certain aspects, the present disclosure provides a method for introducing one or more indels in a cell comprising contacting the CIITA sequence (e.g., exon 1 of CIITA) with a Cas protein or a nucleic acid encoding the Cas protein and a ribonucleic acid, wherein the ribonucleic acid directs Cas protein to and hybridizes to a target motif of the target CIITA polynucleotide sequence, wherein one or more indels are introduced into exon 1 of the CIITA polynucleotide sequence, and wherein the ribonucleic acid has a sequence of SEQ ID NO: 7. In some aspects exon 1 of CIITA is targeted with the ribonucleic acid consisting of SEQ ID NO: 7 and at least one ribonucleic acid or at least one pair of ribonucleic acids selected from the group consisting of SEQ ID NOs: 1-2.

### Insertion of Tolerogenic Factors

In certain embodiments, one or more tolerogenic factors can be inserted or reinserted into genome-edited stem cell lines to create immune-privileged universal donor stem cells. In certain embodiments, the universal stem cells disclosed herein have been further modified to express one or more tolerogenic factors. Exemplary tolerogenic factors include, without limitation, one or more of HLA-G, PD-L1, and CD47. The expression of such tolerogenic factors may inhibit immune rejection.

The present inventors have used genome editing systems, such as the CRISPR/Cas-assisted homology directed repair (HDR) system, to facilitate the insertion of tolerogenic factors into a safe harbor locus, such as the AAVS1 locus, to actively inhibit immune rejection. In some aspects a donor plasmid comprises a HLA-G expression cassette. In some aspects a donor plasmid comprises a PD-L1 expression cassette. In some aspects a donor plasmid comprises a CD47 expression cassette. In certain aspects a donor plasmid comprises a PD-L1, HLA-G, and CD47 expression cassette. In certain aspects a donor plasmid comprises a PD-L1 and CD47 expression cassette. The donor plasmid comprising an expression cassette may target the AAVS1 locus of a stem cell (e.g., a hypoimmunogenic stem cell). In certain aspects the donor plasmid targets the AAVS1 locus of a hypoimmunogenic stem cell with a ribonucleic acid, wherein the ribonucleic acid has a sequence of SEQ ID NO: 8.

In some aspects, the present disclosure provides a stem cell (e.g., hypoimmunogenic stem cell) or population thereof comprising a genome in which the stem cell genome has been modified to express HLA-G. In some aspects, the present disclosure provides a method for altering a stem cell genome to express HLA-G. In certain aspects at least one ribonucleic acid or at least one pair of ribonucleic acids may be utilized to facilitate the insertion of HLA-G into a stem cell line.

In some aspects, the present disclosure provides a stem cell (e.g., hypoimmunogenic stem cell) or population thereof comprising a genome in which the stem cell genome has been modified to express PD-L1. In some aspects, the present disclosure provides a method for altering a stem cell genome to express PD-L1. In certain aspects at least one ribonucleic acid or at least one pair of ribonucleic acids may be utilized to facilitate the insertion of PD-L1 into a stem cell line.

In some aspects, the present disclosure provides a stem cell (e.g., hypoimmunogenic stem cell) or population thereof comprising a genome in which the stem cell genome has been modified to express CD-47. In some aspects, the present disclosure provides a method for altering a stem cell genome to express CD-47. In certain aspects at least one ribonucleic acid or at least one pair of ribonucleic acids may be utilized to facilitate the insertion of CD-47 into a stem cell line.

In some aspects, the present disclosure provides a hypoimmunogenic stem cell (e.g., a stem cell modified to have ablated expression of HLA-A, HLA-B, HLA-C, HLA-DP, HLA-DQ, and HLA-DR) or population thereof comprising a genome in which the stem cell genome has been modified to express PD-L1, HLA-G, and CD47. In some aspects, the present disclosure provides a method for altering a stem cell genome to express PD-L1, HLA-G, and CD47.

In some aspects, the present disclosure provides a hypoimmunogenic stem cell (e.g., a stem cell modified to have ablated expression of HLA-A, HLA-B, HLA-C, HLA-DP, HLA-DQ, and HLA-DR) or population thereof comprising a genome in which the stem cell genome has been modified to express PD-L1 and CD47. In some aspects, the present disclosure provides a method for altering a stem cell genome to express PD-L1 and CD47.

### Universal Stem Cells

In certain aspects, the inventions disclosed herein relate to universal stem cells. The universal stem cells may comprise reduced expression of one or more MHC-I and MHC-II human leukocyte antigens and increased or over expression of one or more tolerogenic factors. In certain aspects the universal stem cells are HLA-A^{-/-}, HLA-B^{-/-}, HLA-C^{-/-}, and CIITA^{indel/indel} cells that exhibit increased expression of HLA-G, PD-L1, and CD47.

In some aspects the stem cells (e.g., the universal stem cells) described herein exhibit one or more features. For example, the stem cells retain the differentiation potential, exhibit reduced T cell response, exhibit protection from NK cell response, and exhibit reduced macrophage engulfment.

The universal stem cells may retain pluripotency, perform tri-lineage differentiation, and retain normal karyotype. For example, the universal stem cells may retain expression of one or more of NANOG, OCT4, SSEA3, and TRA-1-60. In some aspects the universal stem cells are differentiated into the three germ layers (e.g., ectoderm, mesoderm, and endoderm) and maintain expression of all lineage markers.

In some aspects the universal stem cells demonstrate reduced T cell-mediated adaptive immune responses. For example, T cells (e.g., CD4⁺ and CD8⁺ T cells) exhibit reduced priming and activation against the universal stem cells. In addition, T cells exhibit reduced cytokine secretion against the universal stem cells. The reduced expression of HLA-I and HLA-II molecules may result in reduced CD4⁺ and CD8⁺ T cell priming against the universal cells. In some aspects, the expression of PD-L1 further suppresses activation of CD8⁺ T cells.

In some embodiments the universal stem cells are protected from NK cell-mediated rejection. The universal stem cells may be protected from NK cell-mediated rejection as a result of HLA-G expression. In some embodiments the universal stem cells exhibit reduced macrophage engulfment. Overexpression of CD47 and/or expression of PD-L1 in the universal cells may minimize or inhibit macrophage engulfment of the universal cells.

### Some Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, kits and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, kits and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The entire teachings of PCT application PCT/US2016/031551, filed on May 9, 2016, are incorporated herein by reference. All other patents and publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The following example illustrates some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXEMPLIFICATION

Therapies utilizing human pluripotent stem cell-derived cells for transplantation have the potential to revolutionize the way diseases are treated. A major obstacle for their clinical translation is the rejection of allogeneic cells by the recipient's immune system. Strategies aiming at overcoming this immune barrier include banking cells with defined HLA haplotypes (Nakajima et al., 2007; Taylor et al., 2005) and the generation of patient-specific induced pluripotent stem cells (iPSCs) (Takahashi et al., 2007; Yu et al., 2007). However, multiple limitations (de Rham and Villard, 2014; Tapia and Scholer, 2016) prohibit the broader use of these approaches and emphasize the need for “off-the-shelf” cell products that can be readily administered to any patient in need. As a first step to generate such a universal stem cell product, ablating HLA class I is necessary to prevent the presentation of cellular peptides to cytotoxic CD8⁺ T cells, given that HLA class I molecules are expressed in virtually all nucleated cells. Moreover, ablation of HLA class II needs to be considered, since they are also highly polymorphic and can be present in certain hPSC-derived donor cell types, in particular in professional antigen presenting cells (APCs) and endothelial cells (ECs) upon IFNγ stimulation (Ting and Trowsdale, 2002). Recently, the power of CRISPR/Cas9 genome-editing system provided a tool to interfere with HLA class I expression in hPSCs or hematopoietic cells by knocking out the accessory chain beta-2-microglobulin (B2M) (Mandal et al., 2014; Mattapally et al., 2018; Meissner et al., 2014; Riolobos et al., 2013; Wang et al., 2015), and to eliminate HLA class II expression by targeting its transcriptional master regulator, CIITA (Chen et al., 2015; Mattapally et al., 2018). However, the deletion of B2M also prevents the surface expression of nonpolymorphic nonclassical HLA class Ib molecules HLA-E and HLA-G, which are required to maintain NK cell tolerance (Ferreira et al., 2017; Lee et al., 1998b). Moreover, it has been found that B2M-deficient cells are still rejected by allogeneic CD8⁺ T cells (Glas et al., 1992). Therefore, individual deletion of the HLA-A/-B/-C genes may represent a more favorable strategy to protect the donor cells from CD8⁺ T cell-mediated cytotoxicity without losing HLA class Ib protective function.

Other approaches that have been explored to create “off-the-shelf” cell products include the expression of co-inhibitory molecules and the blocking of costimulatory signals required for full T cell activation beyond HLA-T cell receptor (TCR) engagement. For example, ectopic expression of the T cell checkpoint inhibitors PD-L1 and CTLA-4Ig has been shown to protect stem cells from rejection in a humanized mouse model (Rong et al., 2014). Yet, this approach left the HLA-barrier intact, which may result in hyperacute rejection of the engrafted cells precipitated by preexisting anti-HLA antibodies (Iniotaki-Theodoraki, 2001; Masson et al., 2007). Moreover, CTLA-4Ig can also impair T regulatory cell (Treg) homeostasis and function, possibly jeopardizing the establishment of operational immune tolerance (Bour-Jordan et al., 2004; Salomon and Bluestone, 2001).

Innate immune cells, such as NK cells and macrophages, serve an important role in priming adaptive immune responses in many contexts, including chronic graft rejection. A major concern associated with B2M deletion is that this strategy renders the donor cells vulnerable to NK cell mediated killing due to “missing self” (Raulet, 2006). Recently, Gornalusse et al. expressed a B2M-HLA-E fusion construct in B2M-deficient cells to overcome NK cell-mediated lysis (Gornalusse et al., 2017). However, this approach does not address NK cells lacking NKG2A, an inhibitory receptor for HLA-E, whose reactivity could still be concerning (Braud et al., 1998a; Pegram et al., 2011). Therefore, HLA-G, an NK cell inhibitory ligand expressed at the maternal-fetal interface during pregnancy, that acts through multiple inhibitory receptors (Ferreira et al., 2017; Pazmany et al., 1996), might be a better candidate to fully overcome NK cell responses. Moreover, macrophages, which contribute to rejection of transplanted cells, may be controlled by expression of CD47, a “don't-eat-me” signal that prevents cells from being engulfed by macrophages (Chhabra et al., 2016; Jaiswal et al., 2009; Majeti et al., 2009). However, this approach has not yet been explored to protect hPSCs and their differentiated derivatives from macrophage engulfment. Furthermore, a convincing strategy to target both adaptive and innate immunity is yet to be proposed.

Here, it is demonstrated that the CRISPR/Cas9 system can be used to selectively excise the genes encoding the polymorphic HLA class I members, HLA-A/-B/-C, from the genome of hPSCs. Moreover, its multiplexing capacity allows for the simultaneous ablation of HLA class II gene expression using a single guide RNA targeting CHTA. The resulting polymorphic HLA-deficient, “immune-opaque” cells were further modified to express the immunomodulatory factors PD-L1, HLA-G and CD47, which target immune surveillance by T cells, NK cells, and macrophages, respectively, further muting alloresponses in vitro and in vivo. Combining these and other genetic modifications may ultimately result in universal “off-the-shelf” cell products suitable for transplantation into any patient.

Results

### Genome Editing Ablates Polymorphic HLA-A/-B/-C and HLA Class II Expression

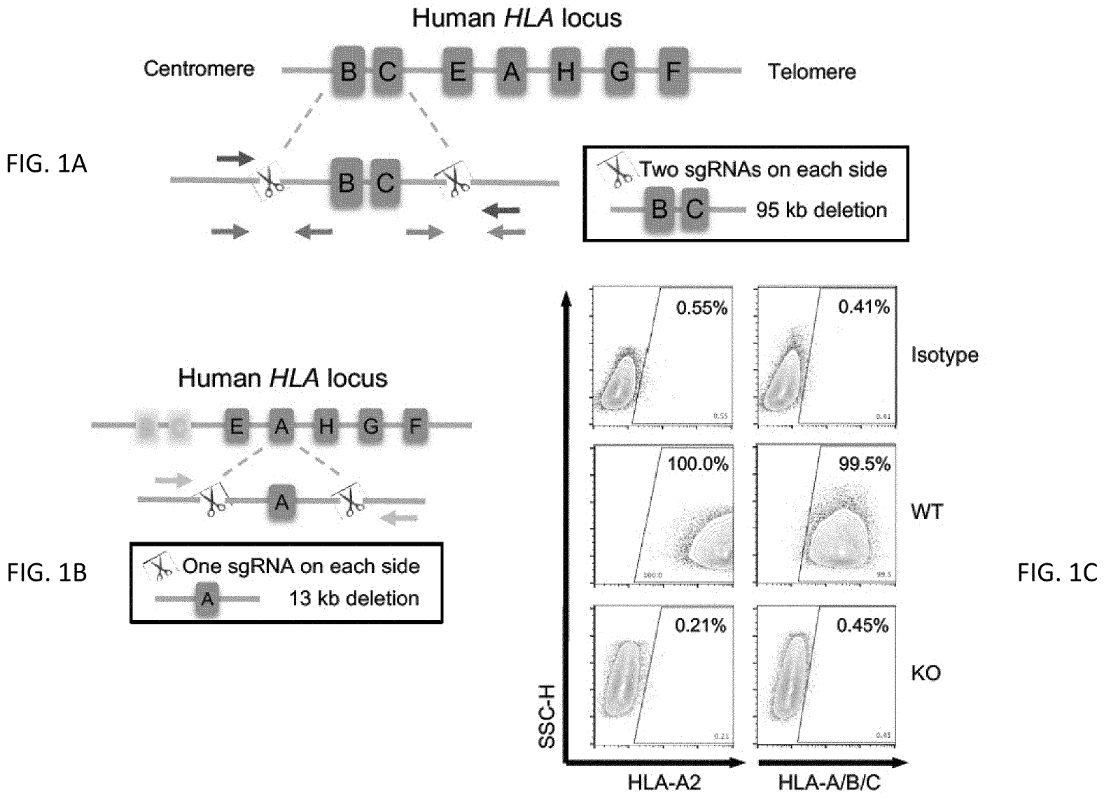

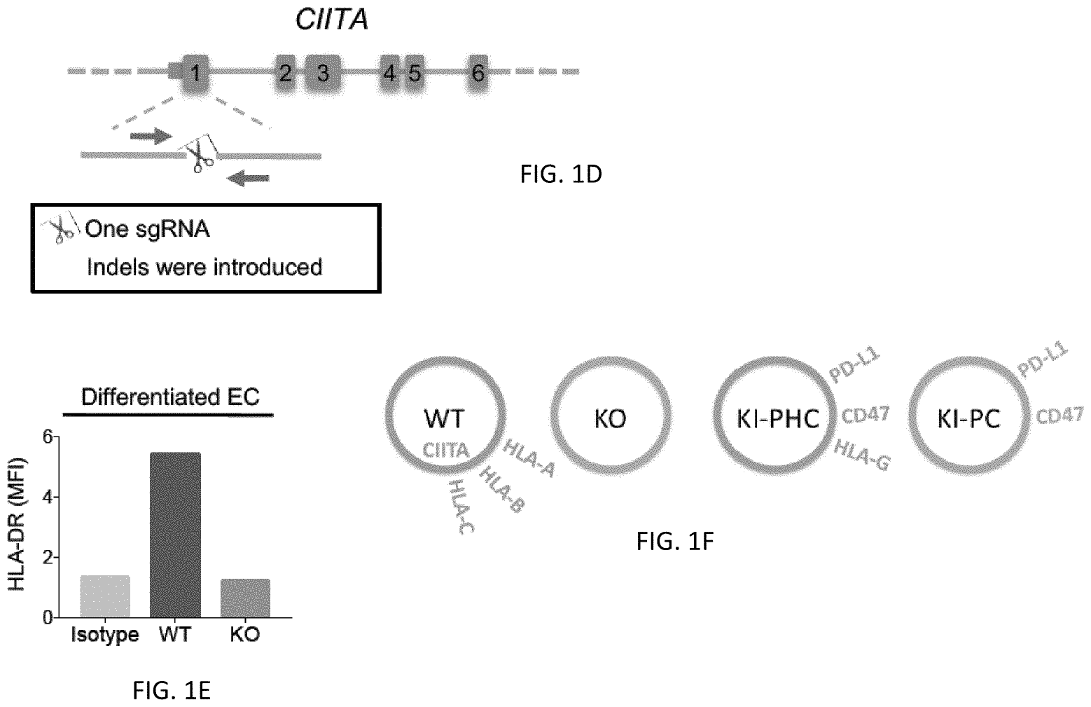

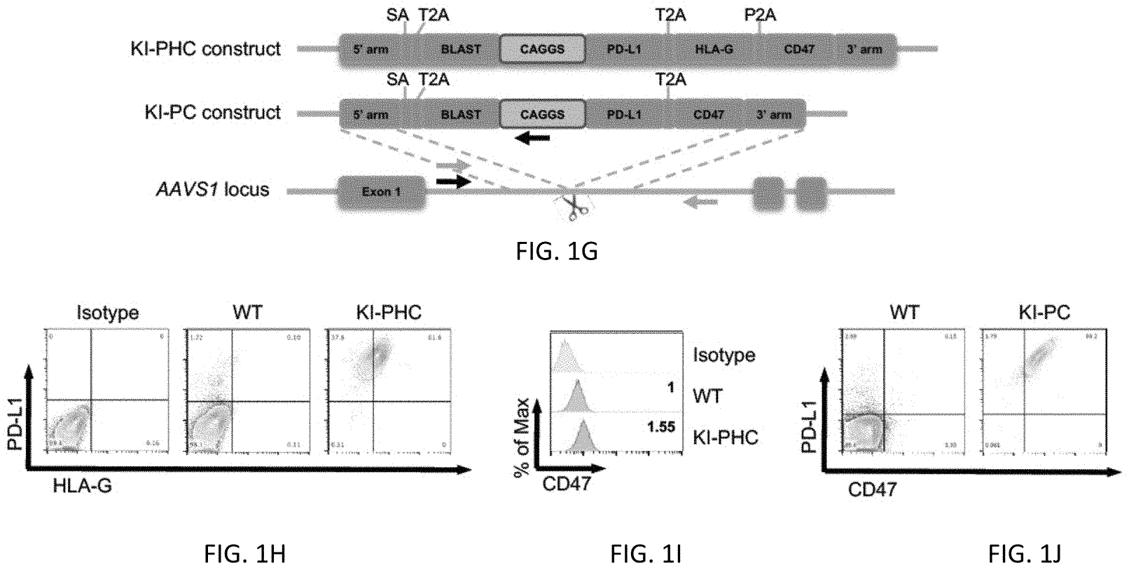

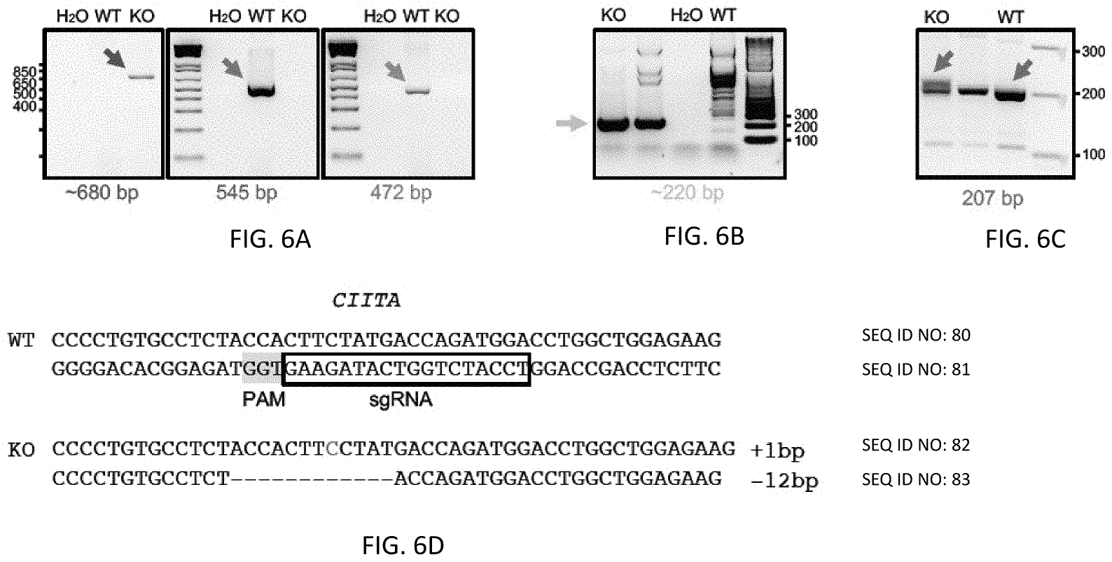

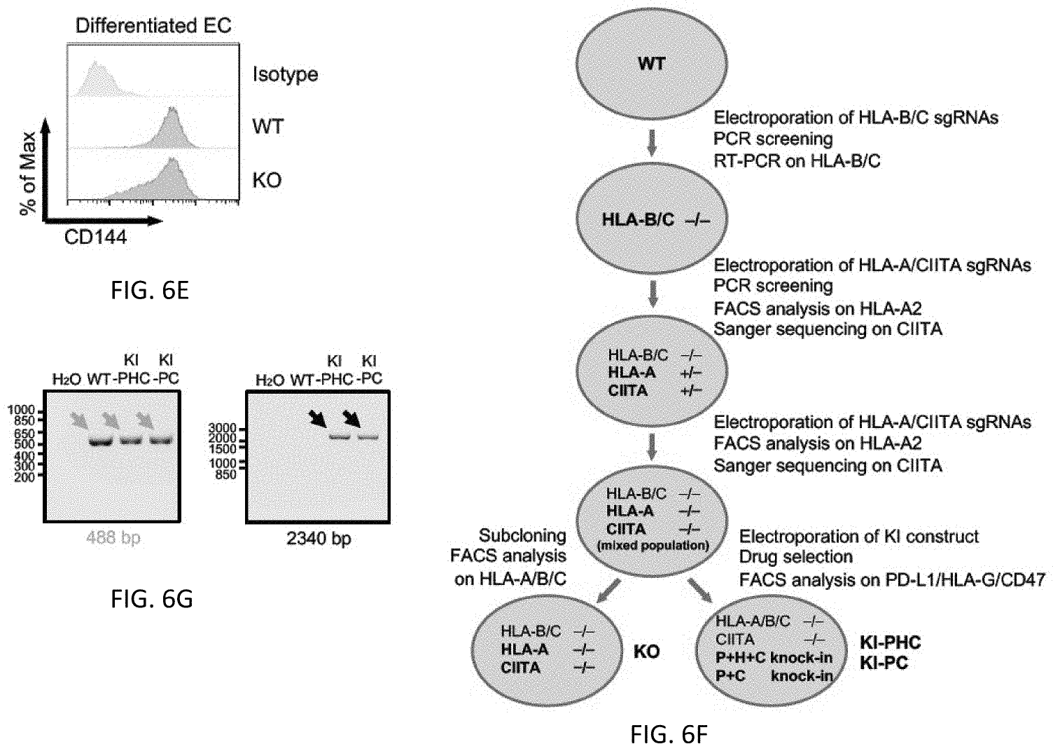

Given that the human MHC class I genes HLA-A, HLA-B, and HLA-C are highly homologous, designing specific short guide RNAs (sgRNAs) targeting the coding regions of each gene using the CRISPR/Cas9 genome-editing system proved challenging. Thus, a dual guide multiplex strategy was employed targeting non-coding regions adjacent to these genes to simultaneously excise all three from the genome of an hPSC line (HUES8). In the HLA locus, HLA-B and HLA-C are adjacent, whereas HLA-A is located nearer the telomere. To simultaneously knock out the adjacent HLA-B and HLA-C genes, two sgRNAs were designed at each site, upstream of HLA-B and downstream of HLA-C(FIG. 1A). The predicted 95 kb deletion also includes the promoters of the two genes, defined as H3K27Ac-positive areas on the UCSC Genome Browser. To knock out the entire HLA-A gene, one sgRNA was designed upstream and another sgRNA downstream of HLA-A (FIG. 1B). The predicted 13 kb deletion includes the HLA-A promoter, according to the UCSC Genome Browser. Both deletions were confirmed by PCR amplicons spanning the predicted Cas9 cutting sites (FIGS. 6A-6B). Ablation of HLA-A/-B/-C proteins in the final HLA knock-out clone (KO), was verified by flow cytometry (FIG. 1C).

Targeting CIITA, the master regulator of HLA class II expression, is a well-documented strategy to collectively ablate the expression of the three highly polymorphic HLA class II alleles, HLADP/-DQ/-DR (Krawczyk and Reith, 2006; Reith and Mach, 2001). A sgRNA targeting exon 1 of CIITA with high cutting efficiency was previously reported (FIG. 1D) (Ding et al., 2013). This sgRNA was used in combination with the sgRNAs targeting the HLA-A gene. A pair of PCR primers flanking the cleavage site in the first exon of CIITA was used to amplify the region spanning the cutting site. PCR amplicons were Sanger sequenced to identify biallelic frame shifts (FIGS. 6C-6D). To demonstrate that targeting CIITA resulted in loss of HLA class II expression, both WT and KO hPSCs were differentiated into endothelial cells (ECs) using a previously published protocol (Patsch et al., 2015). Of note, differentiated WT and KO ECs expressed equivalent levels of the EC marker CD144 (VE-Cadherin), indicating that the differentiation efficiency of the resulting cells was unaffected by genome editing (FIG. 6E). Importantly, induction of HLA-DR expression upon IFNγ stimulation was abolished in KO ECs (FIG. 1E). The KO hPSC clone with a genotype of HLA-A−/−HLA-B−/−HLAC−/−CIITAindel/indel was generated following the workflow depicted in FIG. 6F. Taken together, the results demonstrate that multiplex CRISPR/Cas9 genome editing allows for combined and highly specific ablation of polymorphic HLA class I and II gene expression in hPSCs.

Knock-in of Immunomodulatory Factors into HLA Knockout Cell Line

It was hypothesized that ablating the polymorphic HLA class Ia and class II molecules would eliminate T cell-mediated adaptive immune rejection. However, HLA knockout cells would likely still be susceptible to innate immune cells involved in an alloresponse, such as NK cells and macrophages, prompting the exploration of the effect of introducing immunomodulatory factors based on the following rationale: 1) while the non-polymorphic HLA-E gene will be left intact, its surface expression will likely be severely impaired by the removal of polymorphic HLA class I genes, as the predominant peptides presented by HLA-E are leader peptides derived from other class I molecules (Braud et al., 1998b). Thus, failure to express any HLA class I other than HLA-E may render donor cells vulnerable to NK cell-mediated lysis. To protect the engineered cells from NK cells, it was sought to introduce HLA-G into HLA knockout cells. 2) Macrophages are attracted by cytokines secreted at the site of engraftment and are primed to phagocytose foreign cells by antibody binding. It has been well documented that CD47, which binds to signal regulatory protein alpha (SIRPa) on the surface of macrophages, acting as a “don't eat me” signal, is significantly increased in certain types of tumors and helps them escape macrophage engulfment (Betancur et al., 2017; Jaiswal et al., 2009; Willingham et al., 2012; Zhao et al., 2016). Therefore, it was aimed to overexpress CD47 in HLA knockout cells. 3) HLA-G can present classical peptides derived from intracellular proteins to T cells (Diehl et al., 1996), which would potentially re-expose the cell lines to CD8+ T cell immune surveillance. Furthermore, γδT cells can directly recognize antigens and initiate a cytotoxic response (Vantourout and Hayday, 2013). To counteract any residual T cell response, it was decided to knock in PD-L1, a T cell checkpoint inhibitor that engages the PD-1 receptor on activated T cells, directly suppressing T cell activities (Riley, 2009). Moreover, PD-L1 expression may also contribute to protecting transplanted cells from innate immune rejection by inhibiting PD-1+NK cells (Beldi-Ferchiou et al., 2016; Della Chiesa et al., 2016) and PD-1+ macrophages (Gordon et al., 2017).

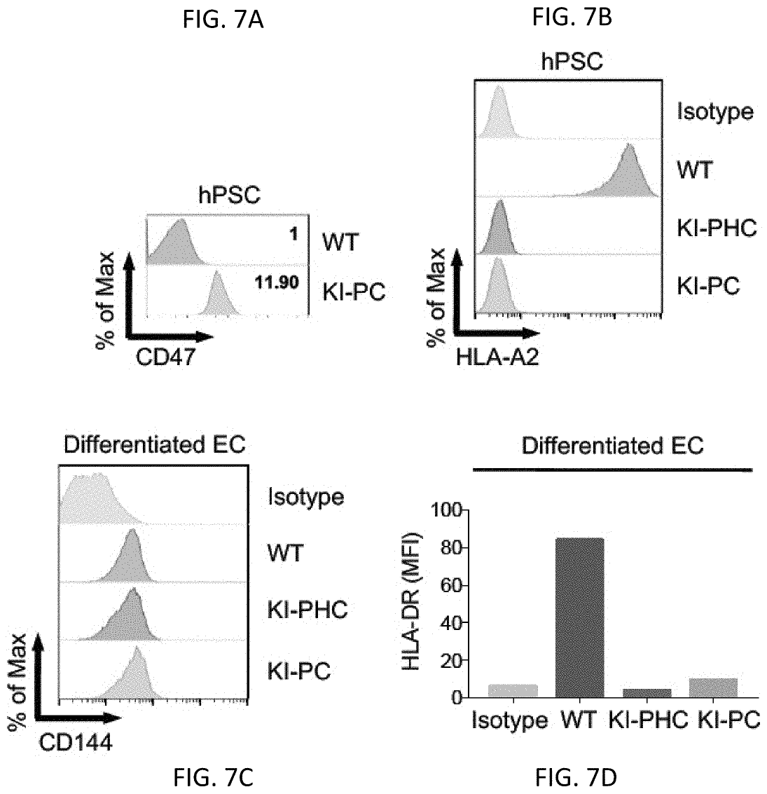

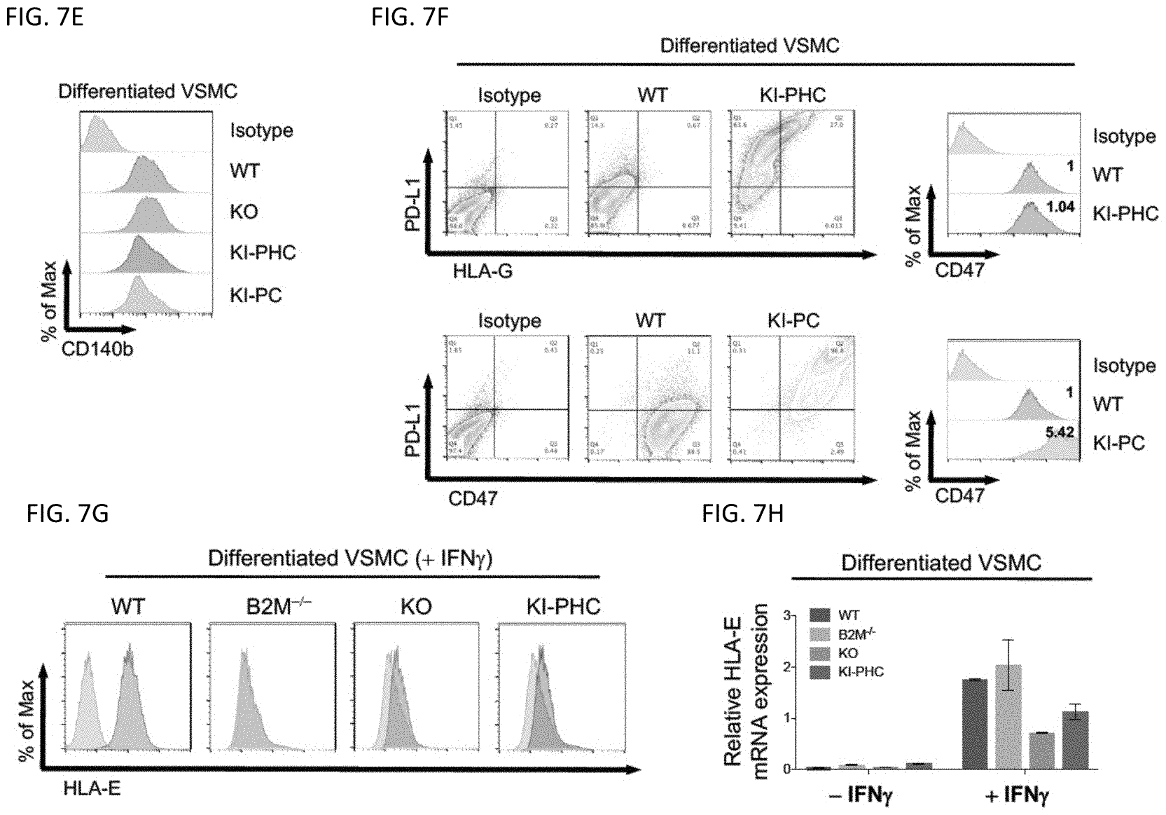

To avoid random integration and positional effects on transgene expression, it was sought to knock-in the immunomodulatory factors into the AAVS1 safe harbor locus (Sadelain et al., 2011). Two donor plasmids were designed, one containing a PD-L1; HLA-G; CD47 expression cassette and another one containing a PD-L1; CD47 expression cassette, both driven by a CAGGS promoter flanked by arms homologous to the AAVS1 locus (FIG. 1G). The donor plasmids were electroporated together with a sgRNA targeting the AAVS1 locus into the HLA-A−/−HLAB−/−HLA-C−/−CIITAindel/indel clone. Integration of the expression cassettes into the AAVS1 locus was verified by PCR (FIG. 6G). Two clones were isolated following the workflow in FIG. 6F and analyzed by flow cytometry; one named KI-PHC that expressed PD-L1, HLA-G, but did not significantly overexpress CD47, compared to WT cells (FIGS. 1H-1I), and a second one named KI-PC that expressed PD-L1 and displayed elevated CD47 level (FIG. 1J and FIG. 7A). Surface HLA-A2 levels were checked by flow cytometry in both KI clones and confirmed HLA class Ia ablation (FIG. 7B). KI-PHC and KI-PC hPSCs were differentiated into CD144+ ECs (FIG. 7C), and no HLA-DR expression was observed by flow cytometry following IFNγ stimulation (FIG. 7D). Thus, immunomodulatory factors were successfully inserted into the AAVS1 safe harbor locus of HLA class Ia and II null cells. Altogether, three engineered hPSC lines: KO, KI-PHC, and KI-PC (FIG. 1F) were generated.

Next, it was sought to confirm the transgene expression as well as HLA-E expression in derivatives of the engineered hPSC lines. For this purpose, the engineered hPSCs were differentiated into vascular smooth muscle cells (VSMCs). WT, KO, KI-PHC and KI-PC VSMCs expressed equivalent levels of the VSMC marker CD140b (PDGFRB), confirming similar differentiation efficiencies (FIG. 7E). In KI-PHC VSMCs, a subpopulation with modestly higher expression of PD-L1 and HLA-G was observed, compared to WT VSMCs, and a major population displaying significantly elevated levels of PD-L1 and HLA-G (FIG. 7F). However, increased CD47 expression in KI-PHC VSMCs was not observed (FIG. 7F), which could be a result of incomplete expression from the targeting cassette, where all three gene products are linked by a 2A-peptide (FIG. 1G). Similarly, a small subpopulation with modestly higher, and a major population with highly elevated levels of PD-L1 and CD47 in KI-PC VSMCs was observed, compared to WT VSMCs (FIG. 7F).

When WT VSMCs were stimulated with IFNγ, they drastically upregulated HLA-E surface expression. In contrast, HLA-E protein levels on the cell surface were greatly reduced in KO VSMCs (FIG. 7G), which was not due to an impaired HLA-E gene expression in KO VSMCs (FIG. 7H). Surprisingly, surface HLA-E expression of KI-PHC VSMCs was not restored by HLA-G expression (FIG. 7G). Nevertheless, HLA-G surface trafficking was unimpaired in the KI-PHC VSMCs (FIG. 7F), providing further incentive to introduce this tolerogenic factor into the engineered cell products to compensate for the reduction of HLA-E surface expression in an HLA-A/-B/-C null background.

### KO and KI Cell Lines Retain Pluripotency and Differentiation Potential

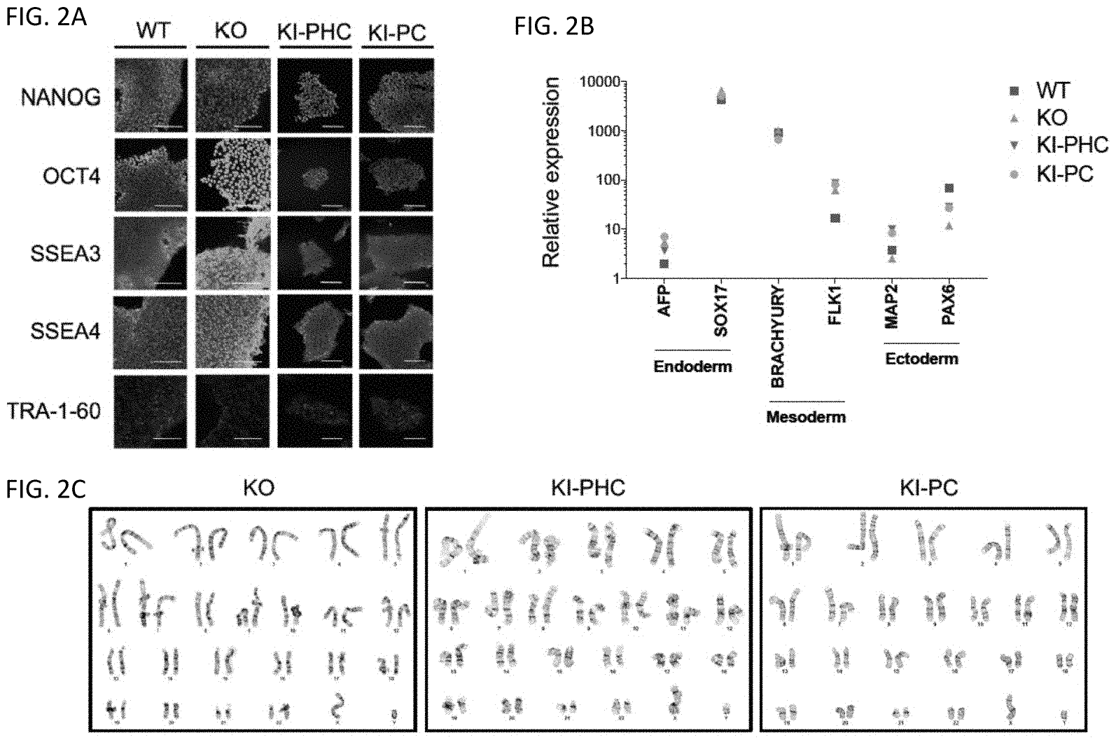

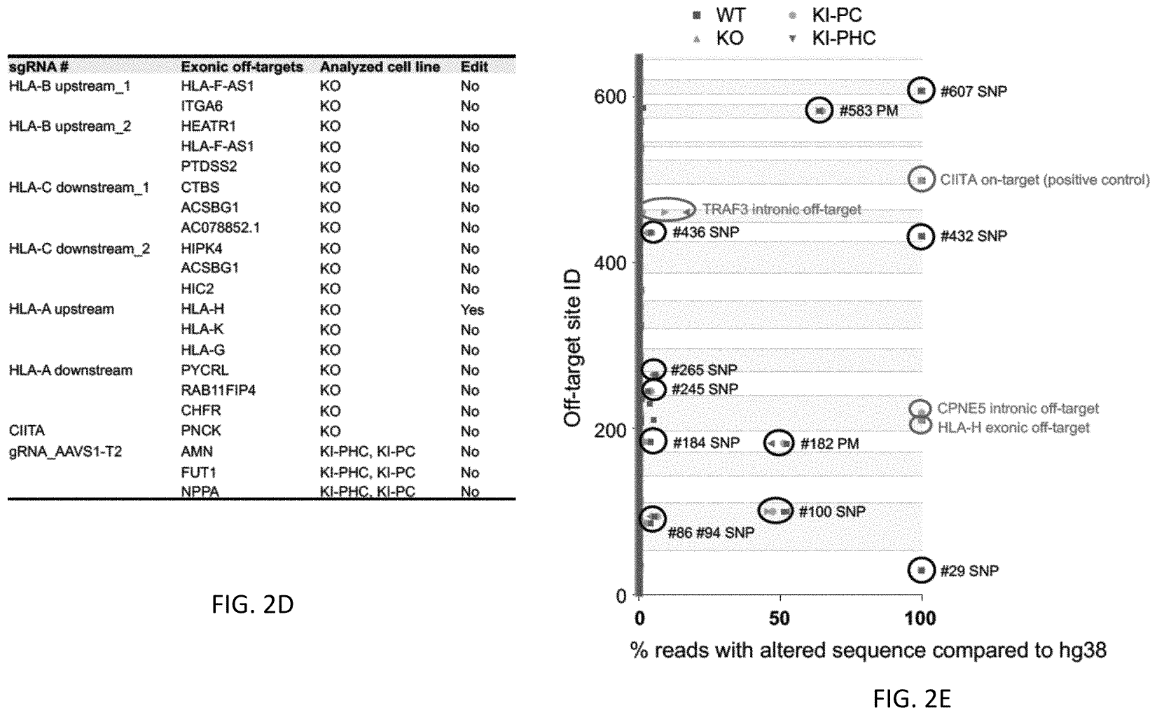

To assess whether the engineered hPSC lines retained pluripotency, expression of NANOG, OCT4, SSEA3, SSEA4, and TRA-1-60 was assessed by immunofluorescence on KO, KI-PHC and KI-PC hPSCs and found equivalent to that of unmodified hPSCs (FIG. 2A). In addition, KO, KI-PHC and KI-PC hPSCs were differentiated into the three germ layers. qRT-PCR was carried out to examine the expression of ectoderm, mesoderm, and endoderm markers and compared to the three germ layers derived from unmodified hPSCs. All of the lineage markers analyzed were found expressed in their respective germ layer cells (FIG. 2B). In addition, the KO, KI-PHC, and KI-PC hPSCs displayed a normal karyotype (FIG. 2C). Thus, despite multiple rounds of genetic modification, these engineered hPSC lines maintained pluripotency, performed tri-lineage differentiation, and retained a normal karyotype.

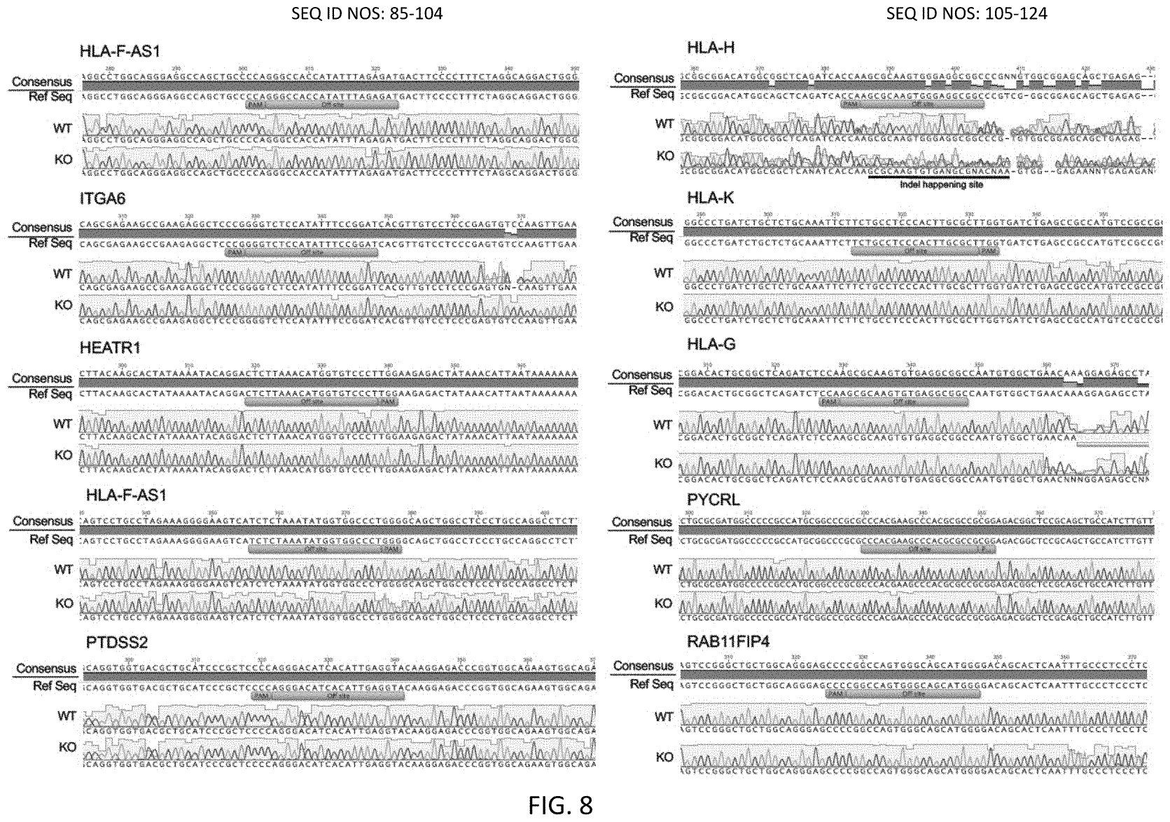

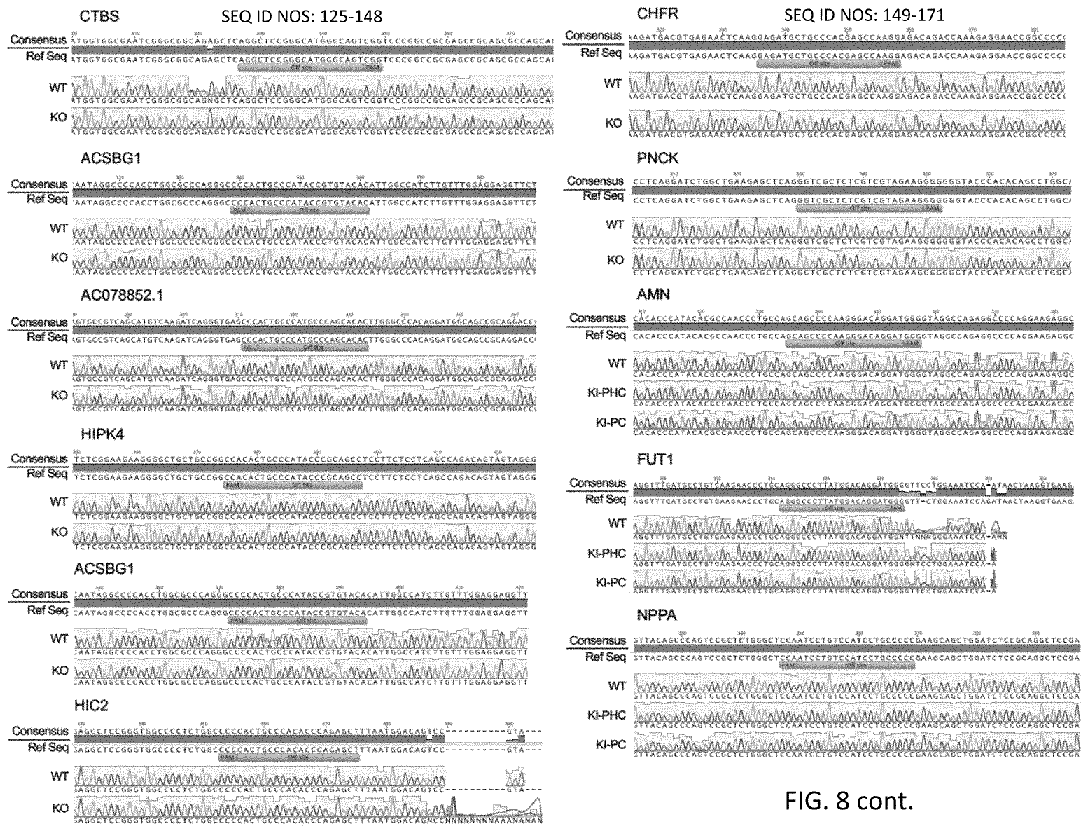

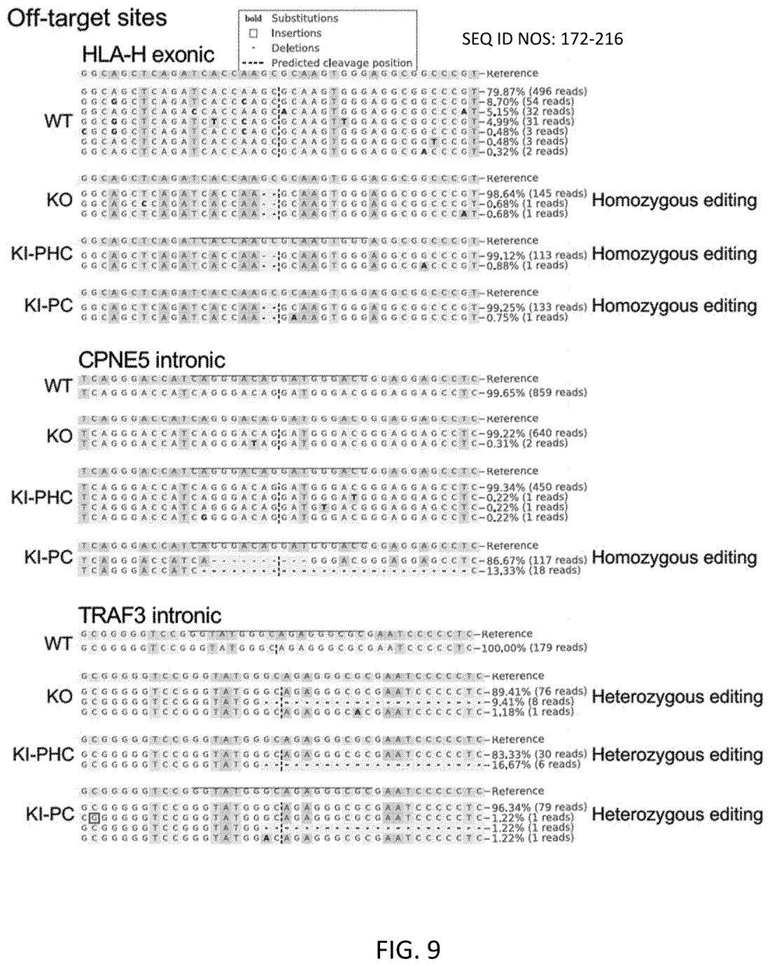

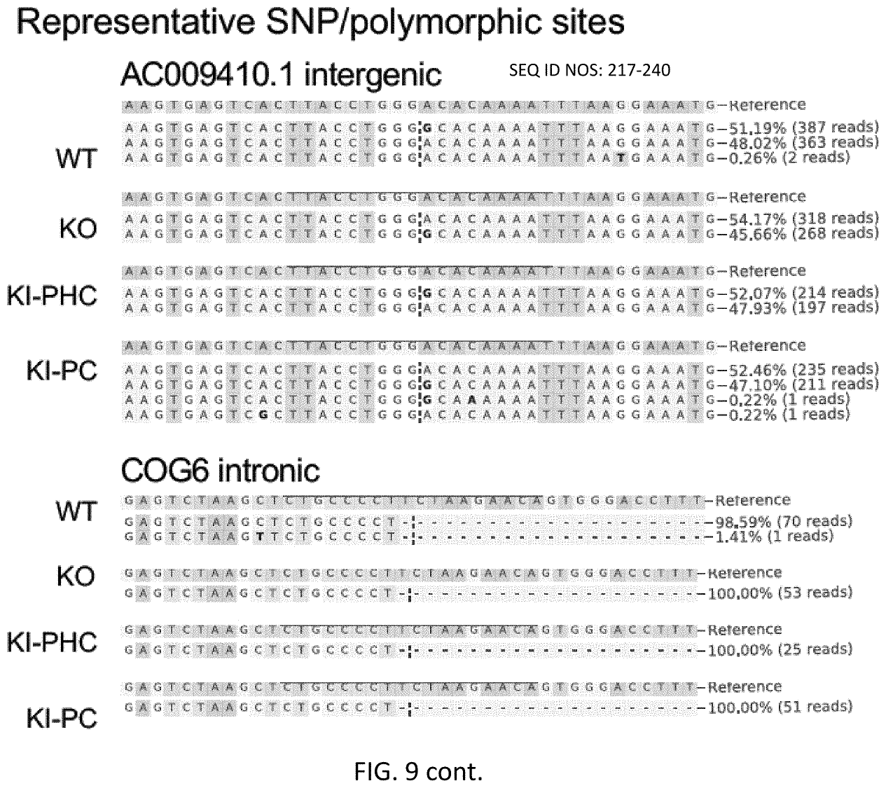

To analyze potential off-target effects of the sgRNAs used to engineer the hPSC lines, the 21 top ranked in silico predicted exonic off-target sites were PCR amplified from the engineered hPSC lines as well as from the parental WT hPSCs. Sanger sequencing of the PCR products did not reveal any unwanted edits on these sites except for the pseudogene HLA-H (HFE), which displayed a perfect match to the sgRNA upstream of HLA-A used to delete HLA-A from the genome (FIG. 2D and FIG. 8). More extensively, target capture sequencing was performed for all of the 648 predicted off-target sites for the eight sgRNAs used in this study. Following enrichment by specifically designed RNA baits, for each predicted off-target site, the enriched DNA fragments were sequenced by next generation sequencing (NGS). Sequence reads of each cell line were aligned and compared to the hg38 genomic reference sequence, and the percentages of reads with altered sequences were calculated. As a result, besides 12 naturally occurring SNP/polymorphic sites identified, HLA-H (HFE) was confirmed as an off-target in all three cell lines. Moreover, an intronic off-target site was detected in TRAF3 in all three cell lines resulting from targeting HLA-C, as well as an intronic off-target site in CPNE5 in the KI-PC cell line as a result of the AAVS1 sgRNA (FIG. 2E, FIG. 9, and Supplementary table 1). Altogether, although three off-target events were detected, the engineered hPSC lines retained pluripotency and their capacity to differentiate into cells of all three germ layers, as well as into VSMCs and ECs with similar differentiation efficiencies to their WT counterparts.

### Reduced T Cell Responses Against KO and KI Cell Lines

Given that removing polymorphic HLA class Ia expression is expected to eliminate T cell-mediated adaptive immune responses, it was next sought to investigate T cell activities in co-cultures with the engineered cell lines. In addition to the KO cells, the KI-PHC cells were also used to address whether the expression of the T cell checkpoint inhibitor PD-L1 would further suppress T cell activity. Four separate in vitro T cell immunoassays were performed: T cell proliferation, activation, cytokine secretion, and killing assays. Since HLA I expression is modest in hPSCs (de Almeida et al., 2013; Drukker et al., 2002), the engineered as well as WT hPSCs were differentiated into ECs, which express both HLA I and II following IFNγ stimulation, or into VSMCs, which only express HLA I, before being used in the respective immunoassays.

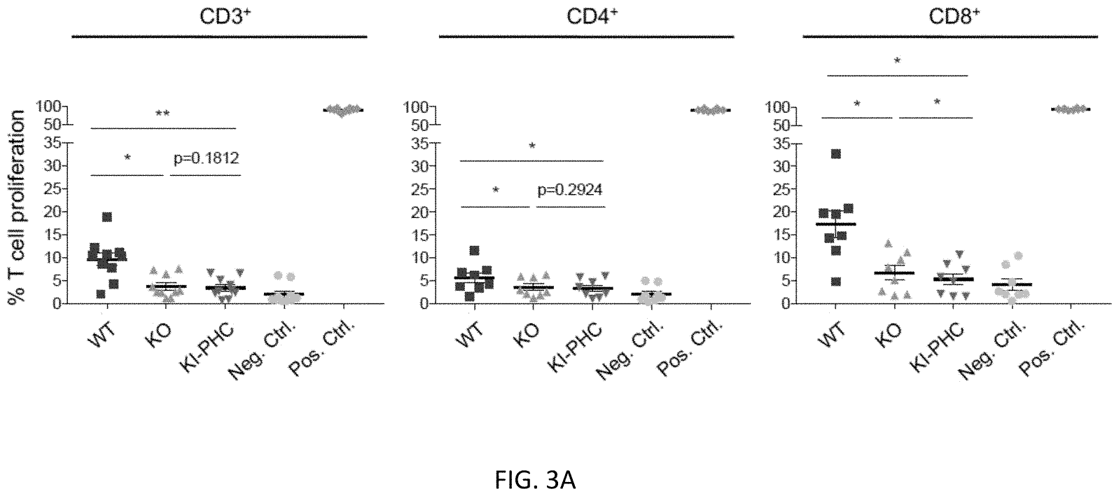

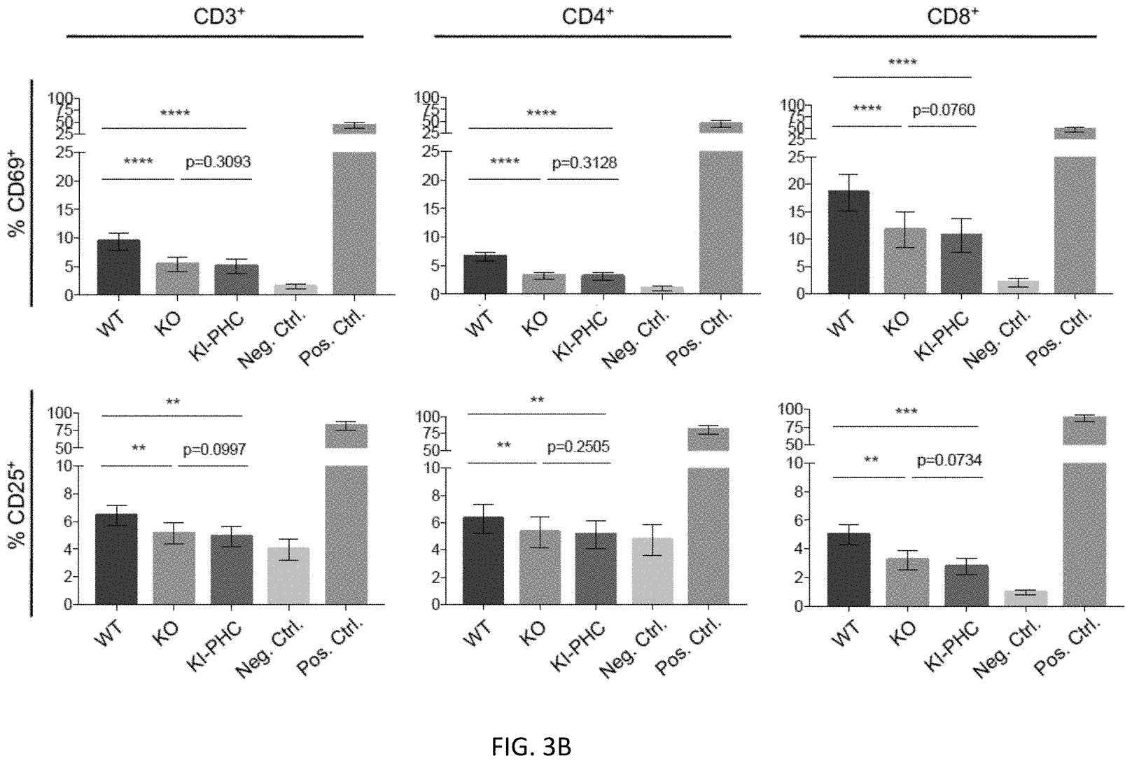

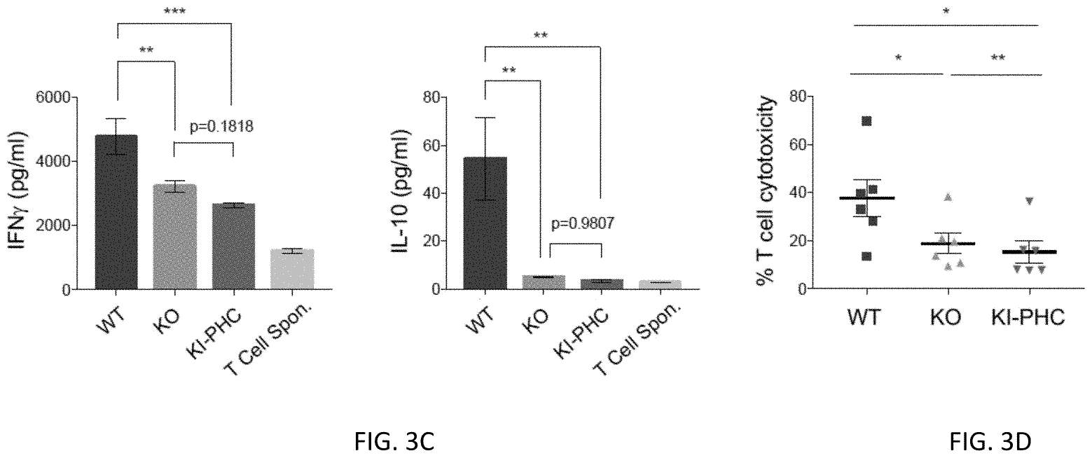

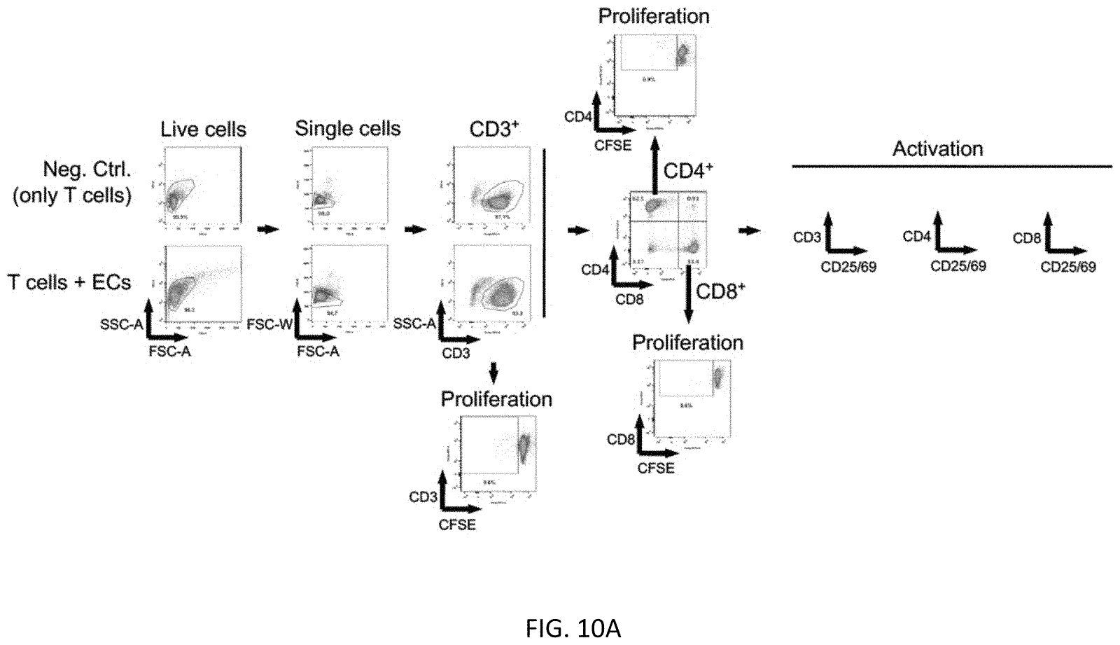

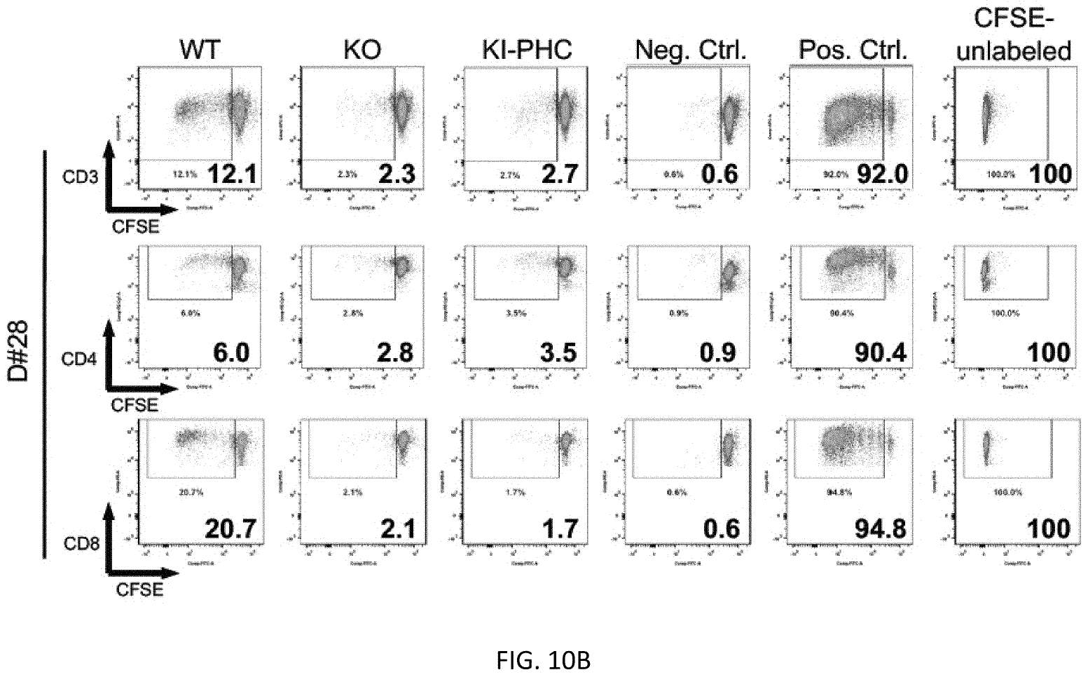

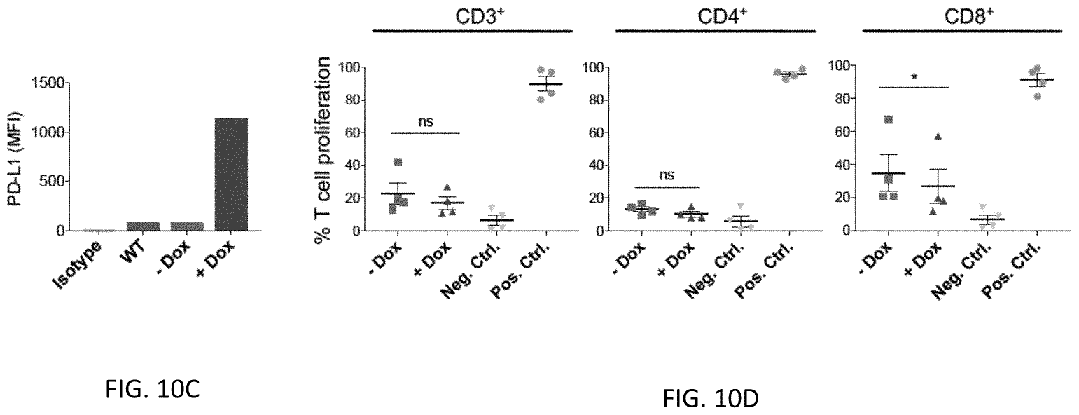

For T cell proliferation assays, WT, KO, and KI-PHC ECs were pre-treated with IFNγ for 48 hours and subsequently co-cultured with CFSE-labeled allogeneic CD3+ T cells for five days. T cells were then stained for CD3/4/8 and analyzed for dilution of the CFSE signal by flow cytometry as a read-out for T cell proliferation in the different T cell subpopulations (FIG. 10A). FACS plots of one representative T cell donor are shown in FIG. 10B. As predicted, the percentage of total proliferating T cells (CD3+) was reduced when incubated with KO ECs (4.17%±0.89% SEM) or KI-PHC ECs (3.87%±0.73% SEM), compared to WT ECs (8.29%±1.23% SEM) (FIG. 3A, left panel). CD4+ T cells followed a similar pattern, with WT ECs (5.03%±0.89% SEM) inducing more CD4+ T cell proliferation than KO ECs (3.58%±0.86% SEM) or KI-PHC ECs (3.49%±0.83% SEM) (FIG. 3A, middle panel). Moreover, CD8+ cytotoxic T cells exhibited significantly reduced proliferation when co-cultured with KO ECs (7.71%±1.89% SEM) or KI-PHC ECs (5.95%±1.48% SEM), as compared to WT ECs (14.32%±2.39% SEM) (FIG. 3A, right panel). Importantly, when compared to co-cultures with KO ECs, CD8+ T cells proliferated significantly less in the presence of KI-PHC ECs (FIG. 3A, right panel), indicating that CD8+ T cell activation was suppressed even further by overexpression of PD-L1 in an HLA null background. To further investigate the suppressive role of PD-L1 during the responses of different T cell subpopulations, an inducible PD-L1-expressing hPSC line was generated and differentiated into ECs before conducting a T cell proliferation assay. It was found that only CD8+, not CD4+, T cell proliferation was reduced in the presence of PD-L1-expressing ECs, when compared to WT ECs, arguing for a specific inhibitory effect of PD-L1 on the CD8+ T cell subset (FIGS. 10C-10D).

Utilizing the same co-culture of T cells with ECs as target cells, the expression of the T cell activation markers CD25 and CD69 was examined (FIG. 3B). Reduced percentages were found of CD25+ and CD69+ T cells (CD3+) in co-cultures with KI-PHC ECs (4.91%±0.74% SEM; 5.04%±1.24% SEM) or KO ECs (5.12%±0.77% SEM; 5.40%±1.29% SEM), when compared to T cells co-incubated with WT ECs (6.43%±0.71% SEM; 9.30%±1.51% SEM) (FIG. 3B). The same trends were observed in the CD4+ and the CD8+ cell populations (FIG. 3B). It also found that, when co-cultured with WT ECs, a higher percentage of CD25+ cells was observed in the CD4+ cell population, whereas a higher percentage of CD69+ cells was observed in the CD8+ cell population. However, a significantly reduced expression of activation markers in T cells against KI-PHC ECs when compared to KO ECs was not observed.

Next, the levels of the T cell effector cytokines IFNγ and IL-10 secreted into the medium over the course of a five-day T cell-EC co-culture were examined Compared to the levels of IFNγ and IL-10 observed in media following exposure to WT ECs (4747±556.1 SD; 54.56±17.22 SD), the levels of both cytokines were lower in media when T cells were exposed to either the KO (3214±180.5 SD; 5.09±0.16 SD) or KI-PHC ECs (2635±132.9 SD; 3.56±0.63 SD), indicating reduced cytokine secretion from T cells against KO or KI-PHC cell lines (FIG. 3C).

To quantify T cell killing, lactate dehydrogenase (LDH) released from VSMCs was measured as a surrogate for T cell cytotoxicity. In this setting, only the CD8+ T cells were expected to be activated by HLA I-TCR engagement, given that VSMCs solely express HLA I. It was found that the CD8+ T cell cytotoxicity against KI-PHC VSMCs (15.31%±4.52% SEM) was the lowest when compared to KO (18.86%±4.34% SEM) and WT (37.65%±7.64% SEM) VSMCs (FIG. 3D). This observation suggests that the CD8+ T cell cytotoxicity was suppressed even further by PD-L1 in KI-PHC VSMCs, consistent with the results of the CD8+ T cell proliferation assay. Collectively, the observations in the T cell immunoassays demonstrate reduced CD4+ and CD8+ T cell priming against KO and KI-PHC cell lines as a result of the removal of HLA I and II molecules. CD8+ T cell activation was further suppressed by the expression of PD-L1 in the KIPHC cell line.

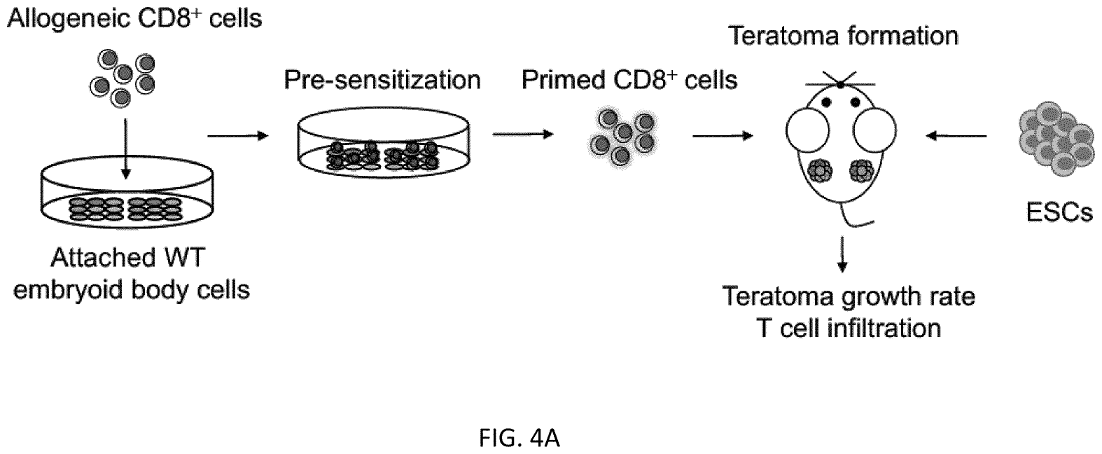

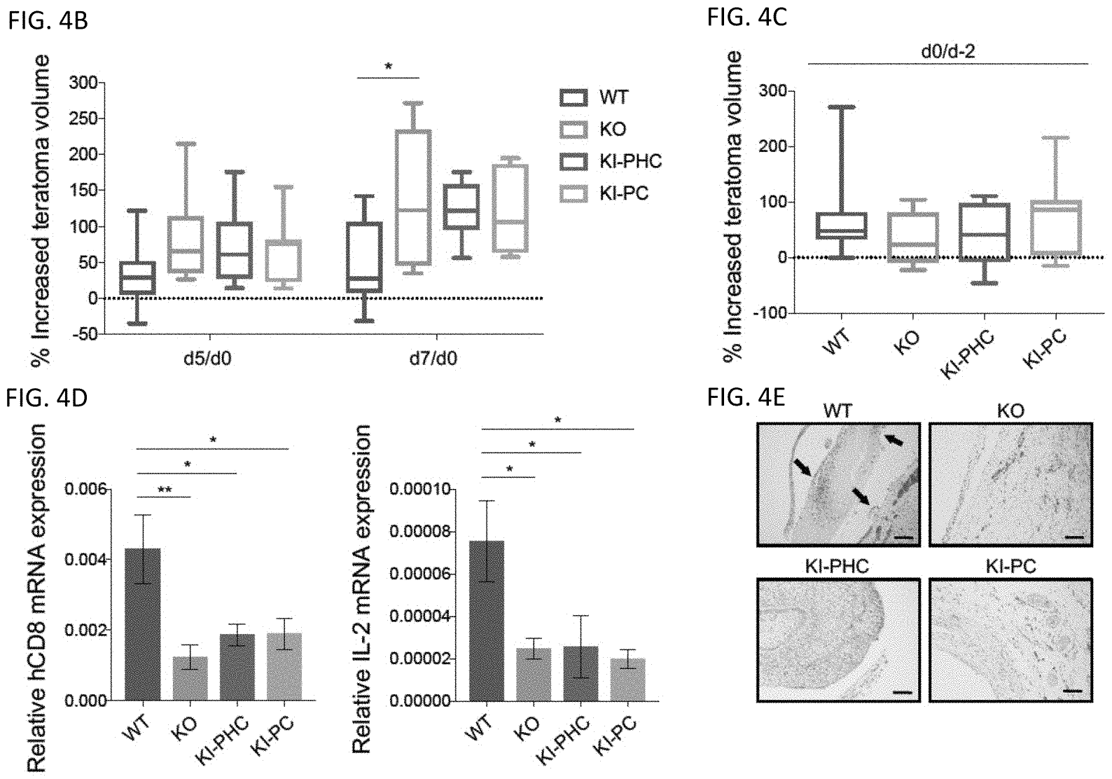

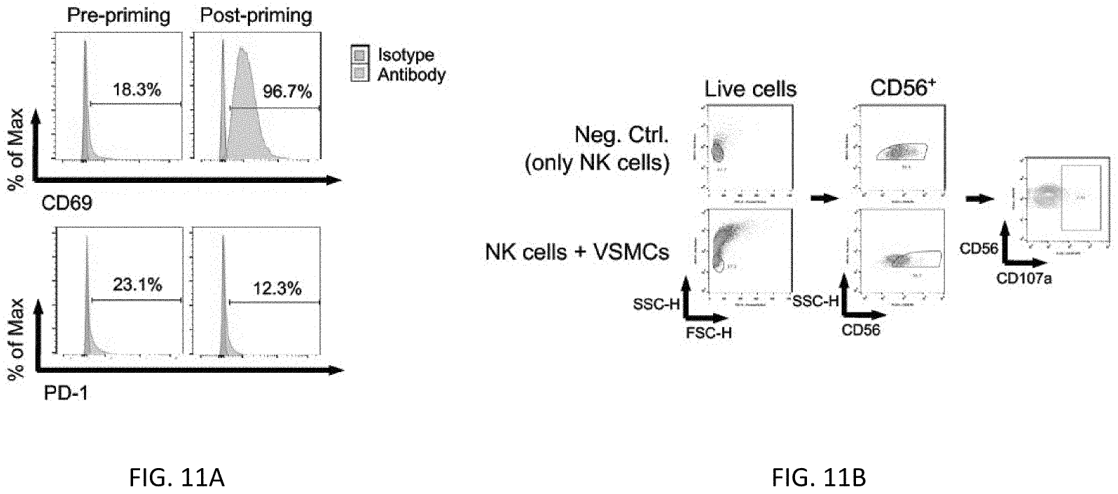

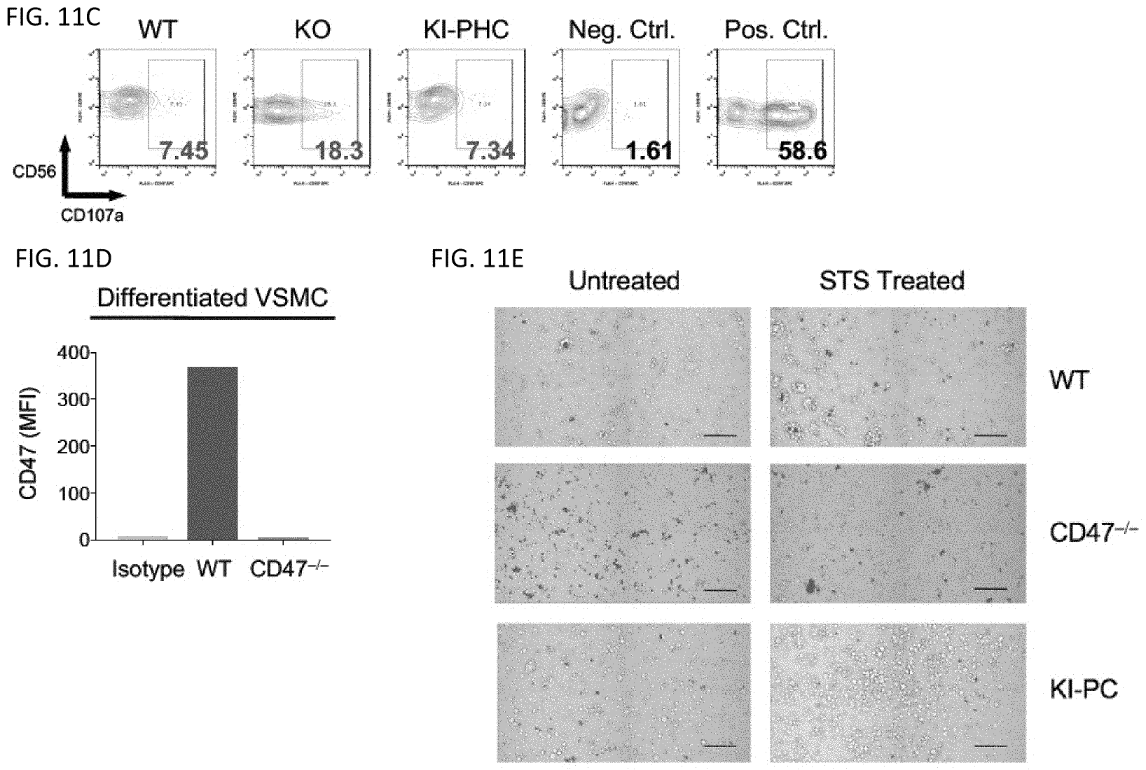

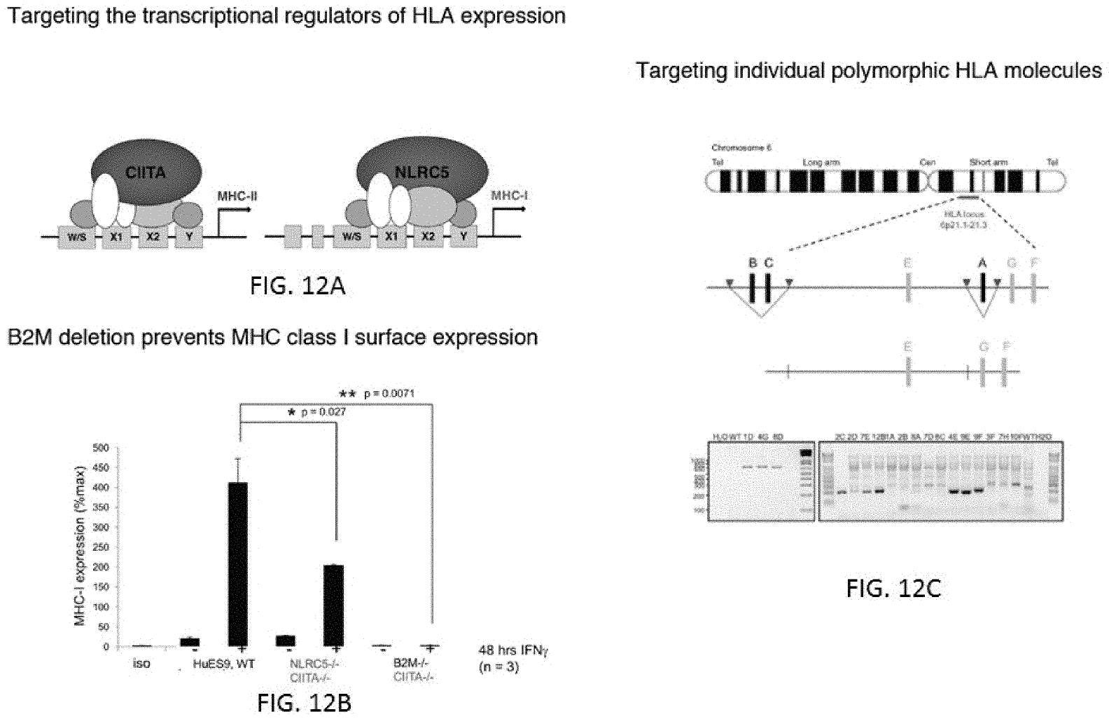

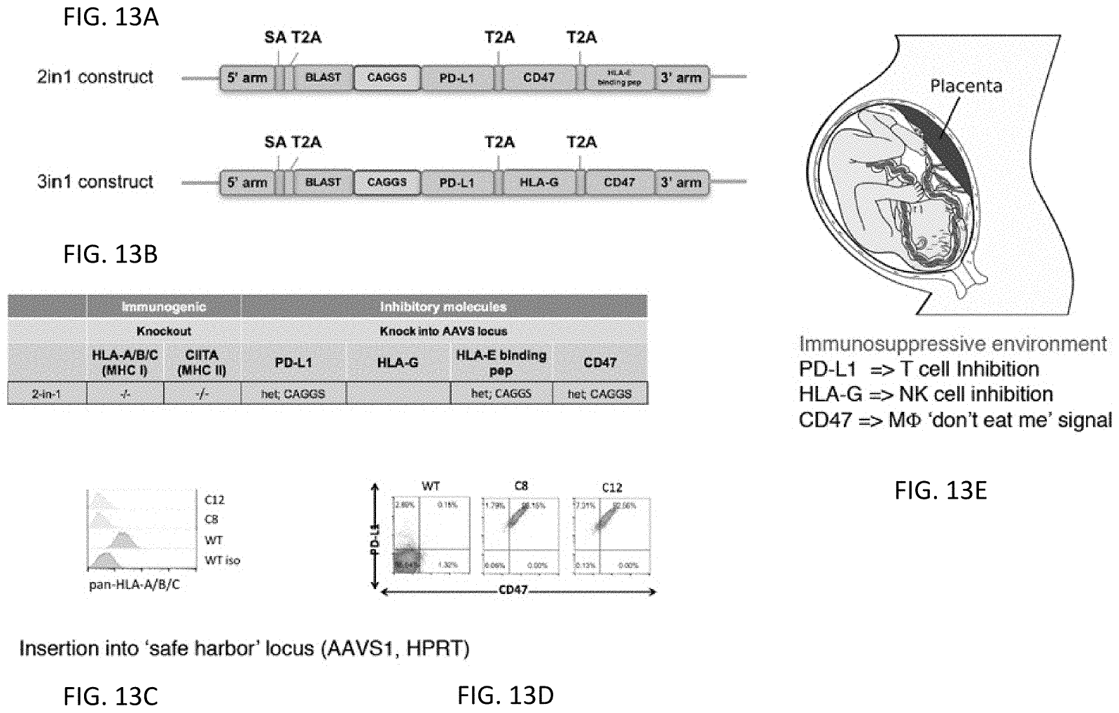

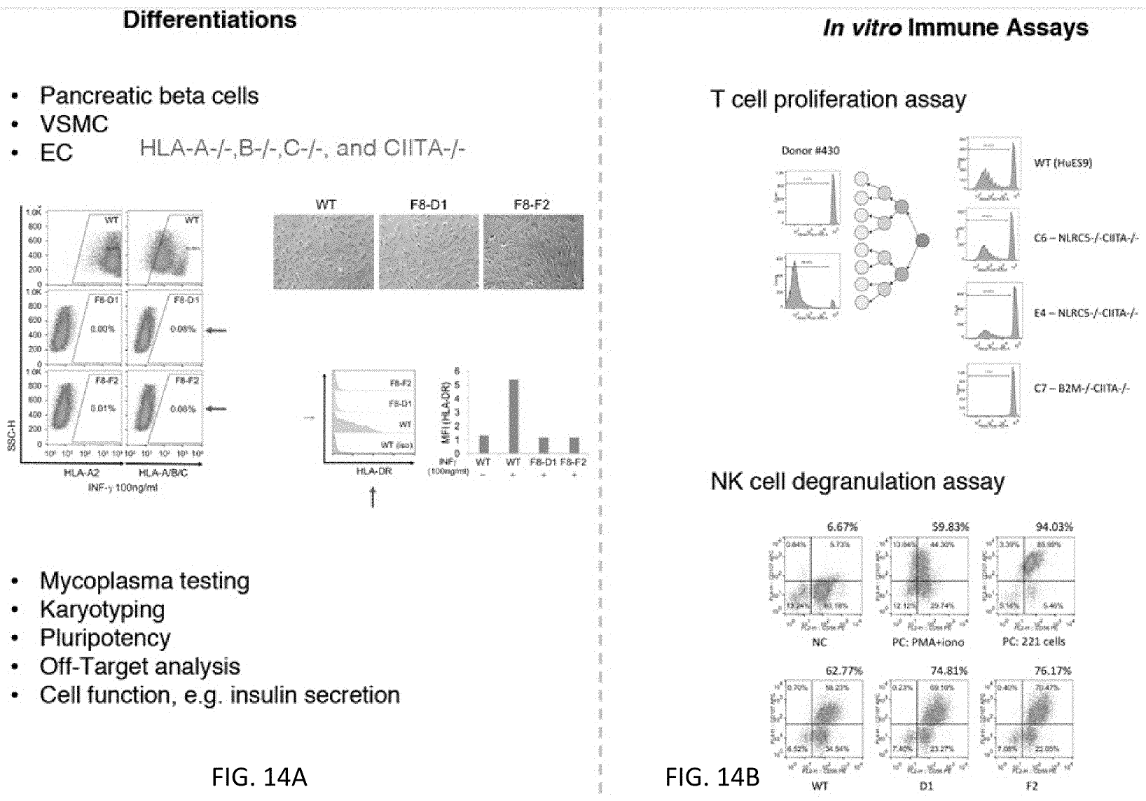

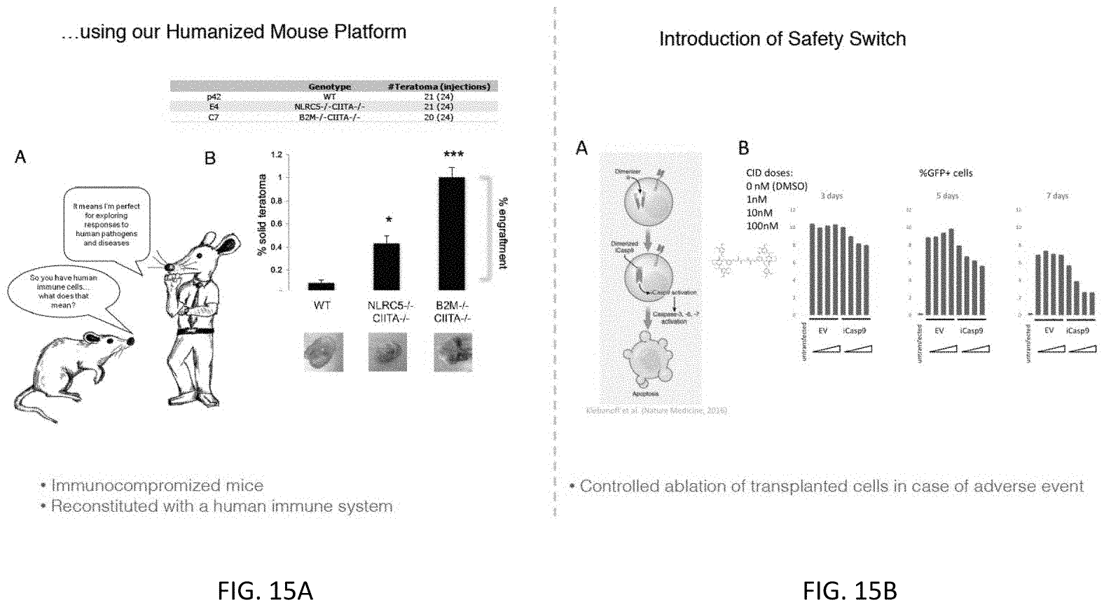

To assess T cell responses in vivo, WT and the engineered hPSCs were transplanted subcutaneously into immunodeficient mice and allowed to form teratomas over the course of 4-6 weeks. Pre-sensitized allogeneic CD8+ T cells were then adoptively transferred via tail vein injection and teratoma growth was monitored for an additional 8 days (FIG. 4A). As measured by CD69 and PD-1 expression of CD8+ T cells pre- and post-priming, the T cells used for injection were activated (CD69+) and without signs of exhaustion (PD-1+) following sensitization (FIG. 11A). In agreement with the hypothesis that only the WT cells will be rejected, WT teratomas, displayed a slower increase in volume compared to KO teratomas seven days after injection of CD8+ T cells, which was not due to a slower growth rate of the WT teratomas themselves (FIGS. 4B-4C). These results suggest that the KO teratomas were protected against T cell-mediated rejection. Moreover, although not significant, the average volumes of the KI-PHC and KI-PC teratomas were also larger than that of the WT teratomas 7 days post T cell infusion (FIG. 4B). In addition, teratomas derived from both, the KO and KI cell lines, displayed reduced T cell infiltration, as evidenced by qPCR for the human effector T cell markers CD8 and IL-2 (FIG. 4D), as well as by histology (FIG. 4E). Together, these observations suggest that removal of the polymorphic HLA molecules from the cell surface of transplanted cells can effectively block T cell-mediated rejection in vivo, matching the in vitro observations.

KI Cell Lines are Protected from NK Cell and Macrophage Responses

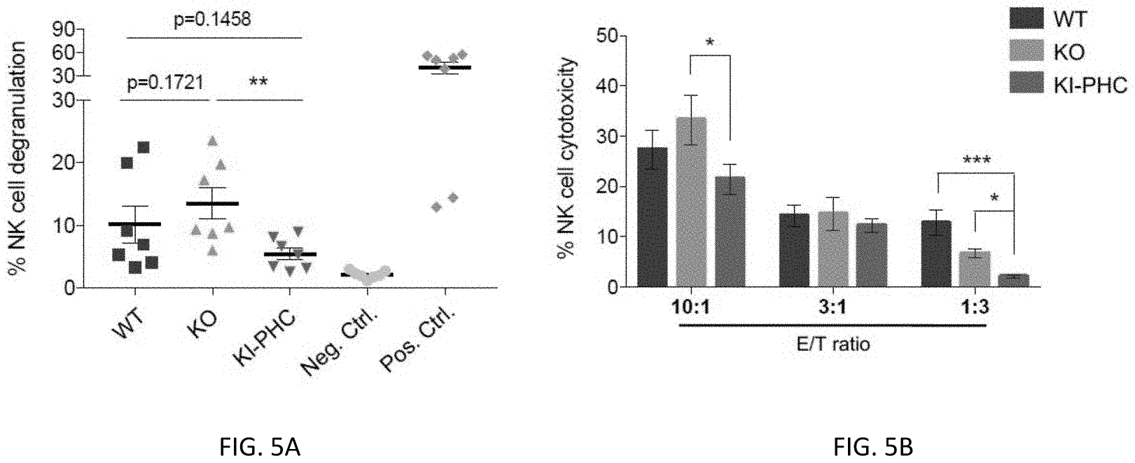

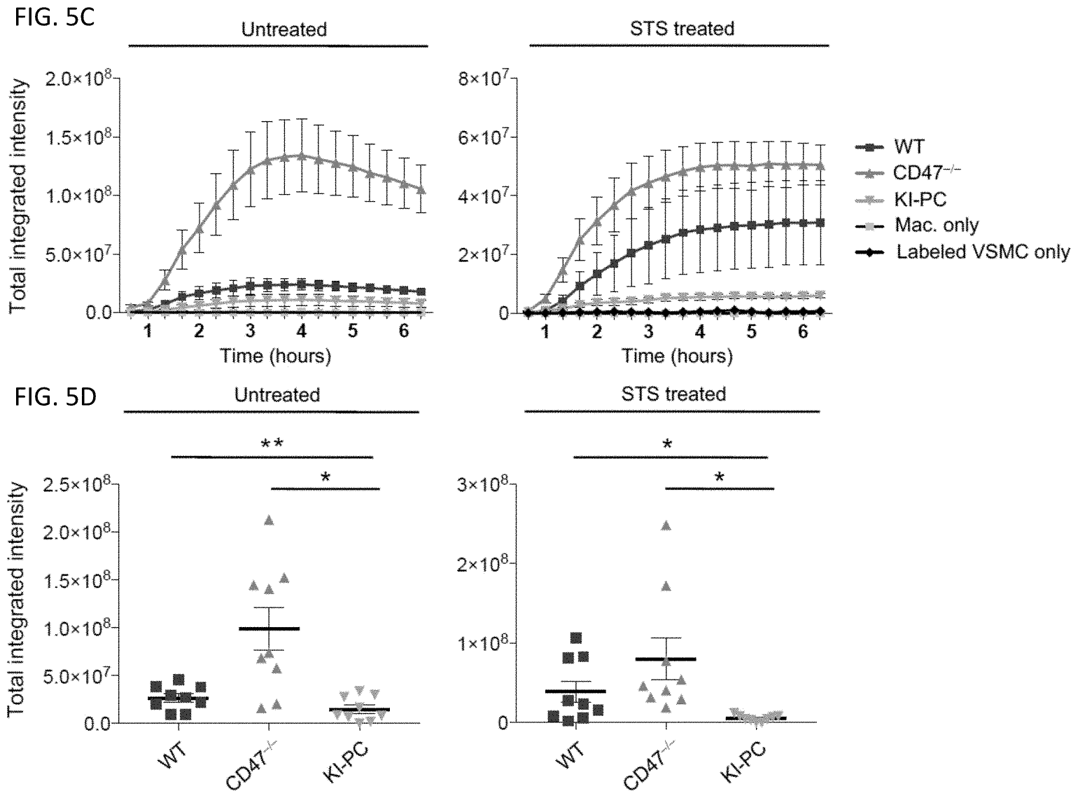

Due to the lack of HLA Ia molecules and impaired HLA-E surface expression, the KO hPSCs and their derivatives were expected to be vulnerable to NK cell-mediated lysis, whereas the KI-PHC cell line should be protected from NK cell-mediated rejection as a result of HLA-G expression. To test the hypothesis, allogenic NK cells were isolated from healthy donors and co-incubated with WT, KO, or KI-PHC VSMCs. CD56+NK cells were analyzed by flow cytometry for surface expression of the degranulation marker CD107a as a readout of NK cell activation (FIG. 11B). Of note, NK cell degranulation in the presence of KO VSMCs was not significantly higher than with WT VSMCs (10.16%±2.96% SEM) (FIG. 5A), suggesting the lack of an NK cell activation signal on hPSC-derived VSMCs. However, in agreement with the hypothesis, it was found that the percentage of CD107a+ degranulating NK cells in a co-culture with KI-PHC VSMCs (5.43%±0.95% SEM) was significantly lower than in the presence of KO VSMCs (13.51%±2.51% SEM) (FIG. 5A), suggesting that NK cell activity is indeed inhibited by HLA-G expression in KI-PHC VSMCs. FACS plots of one representative donor are shown in FIG. 11C. The LDH released from apoptotic VSMCs after coincubation with NK cells was also examined to quantify NK cell cytotoxicity. Consistent with NK cell degranulation, it was observed that NK cell cytotoxicity was reduced when NK cells were incubated with KI-PHC VMSCs (FIG. 5B).

Finally, macrophage activity was examined using a pH-sensitive fluorescent dye (pHrodo-Red) that emits a signal upon phagocytic engulfment. It was hypothesized that overexpression of the macrophage ‘don't-eat-me’ signal CD47 in derivatives of the engineered hPSC cell lines would reduce macrophage engulfment. Given that no significant increase of CD47 expression was observed in KI-PHC VSMCs (FIG. 7F), VSMC differentiated from the KI-PC cell line was used in these assays, which displayed much higher CD47 level than WT VSMC (FIG. 7F). In addition, a CD47 knockout (CD47−/−) cell line was generated as a positive control for macrophage engulfment and verified the loss of CD47 cell surface expression by flow cytometry (FIG. 11D). pHrodo-Red labelled VSMCs differentiated from WT, CD47−/− and KI-PC cells were either treated with staurosporine (STS) to induce apoptosis or left untreated and then incubated with isolated allogeneic macrophages from healthy donors. The emergence of red signal, an indicator of VSMCs that were engulfed by macrophages, was monitored by live cell imaging and the fluorescence intensity was quantified. Of note, with or without STS treatment, KI-PC VSMCs showed significantly decreased engulfment by macrophages when compared to CD47−/− or WT VSMCs (FIGS. 5C-5D, and FIG. 11E). These data demonstrate that overexpression of CD47 can indeed minimize macrophage engulfment of engineered hPSC-derived VSMCs, although a contribution of PD-L1 to inhibiting macrophage engulfment cannot be ruled out, which was also expressed by KI-PC VSMCs (FIG. 7F).

### DISCUSSION

In this study, multiplex CRISPR/Cas9 genome editing was applied to knock out the highly polymorphic HLA-A/-B/-C genes, and successfully prevented the expression of HLA class II genes by targeting the CIITA gene in hPSCs. In addition, CRIPSR/Cas9-assisted homology directed repair (HDR) was used to introduce the immunomodulatory factors PD-L1, HLA-G and CD47 into the AAVS1 locus. It was found that the engineered hPSC derivatives elicited significantly less immune activation and killing by T cells and NK cells and displayed minimal engulfment by macrophages.

In the approach for ablating HLA class Ia expression, the polymorphic HLA class Ia genes, HLA-A/-B/-C were specifically excised, while leaving the genes B2M and the nonpolymorphic HLA class Ib genes HLA-E, -F and -G intact. While the resulting 95 kb deletion contains not only HLA-B/-C genes, but also M1R6891 and four pseudogenes, there were no observed changes in growth rate or differentiation efficiency in the KO or KI cell lines. Interestingly, for unknown reasons, HLA-E surface expression was not restored by the expression of HLA-G in KI-PHC cells, which was inconsistent with a previous report that the leader peptide from HLA-G is sufficient to promote HLA-E surface trafficking (Lee et al., 1998a).

The HLA knockout (KO) hPSC line was generated by genome editing using seven different sgRNAs, and KI-PHC and KI-PC hPSC were clones derived from the KO line and edited by an additional sgRNA targeting the AAVS1 locus. Out of 648 predicted off-target sites for the eight sgRNAs used, only one exonic off-target event was observed in the transcribed pseudogene HLA-H, as a result of one of the sgRNAs used to delete HLA-A from the genome. If translated, the observed 2 bp deletion found in both alleles would result in a frameshift-causing mutation. Even though mutations in the HLA-H have been linked to hereditary hemochromatosis, a rare iron storage disorder (Feder et al., 1996), the observed HLA-H (HFE) mutation did not impact the growth rate or differentiation efficiencies of the cell types tested in this study. Of note, it would be possible to avoid this off-target event, either by designing a different sgRNA or by selecting clones that do not harbor this particular off-target mutation by genotyping the HLA-H locus. Moreover, using sgRNA/Cas9 ribonucleoprotein complexes (RNP) for targeting, which allows for more transient editing than plasmid-based approaches (Roth et al., 2018), should reduce the number of off-target events per cell line and thus be applied in the future.

As expected, the removal of polymorphic HLA expression in hPSCs and their derivatives, such as ECs and VSMCs, resulted in reduced T cell responses in vitro and in vivo. An interesting observation from the T cell assays is that overexpression of the checkpoint inhibitor PD-L1 only had a significant impact on the proliferation and cytotoxicity of CD8+ T cells. This may have several possible explanations: 1) the levels of the PD-L1 receptor, PD-1, are higher on CD8+ T cells than on CD4+ T cells. 2) CD8+ T cells are the cell type most responsive to target cell exposure in the assays and hence will also express higher levels of the negative regulator PD-1. Of note, both explanations are not mutually exclusive, as the strength of T cell activation and PD-1 expression are linked by a negative feedback loop (Riley, 2009). Moreover, it was noted that PD-L1 alone had an impact on CD8+ T cell proliferation even in the absence of HLA, suggesting that PD-L1 can act as a tolerogenic factor even in the absence of a productive HLA-TCR interaction. In addition, in the T cell activation and cytokine secretion assays, when compared to the negative control, background T cell activity was observed even in a co-culture with the KIPHC cell line. This could be due to the experimental setup, considering target cells may secrete factors that promote T cell activation independent of the presence of HLA.

While acute graft rejection is mainly T cell-mediated, the role of other immune cells such as macrophages, NK cells, and B cells must also be considered with regards to engraftment and long-term survival of therapeutic cells. The NK cell assays suggest that HLA-G expression was able to control NK cell activities. Moreover, overexpression of CD47 effectively reduced macrophage engulfment. Yet, as recently reported, PD-L1, which was co-expressed in both KI cell lines, can also impact the activities of PD-1+NK cells (Beldi-Ferchiou et al., 2016; Della Chiesa et al., 2016) and PD-1+ macrophages (Gordon et al., 2017), which may contribute to the observed phenotypes. With regards to long-term engraftment, in particular, antibody-dependent cellular cytotoxicity (ADCC) by NK cells, and allo-antibody-mediated complement activation as the main drivers of chronic graft rejection must be considered (Baldwin et al., 2016; Djamali et al., 2014; Michaels et al., 2003). It can be envisioned that introducing additional factors known to inhibit ADCC and complement activation, such as CD59 (Meri et al., 1990), may enable durable engraftment. Ultimately, in vivo experiments will help clarify the extent of protection that modified cells may have following transplantation. Yet, while various humanized mouse models exist, they are limited in re-capitulating a full human immune response. Thus, the development of improved in vivo models for testing cell transplantation and rejection may be required (Brehm et al., 2014; Li et al., 2018; Melkus et al., 2006; Rongvaux et al., 2014).

Overcoming the immune barrier to transplantation would provide an exciting new modality not only to overcome the allobarrier, but also potentially to treat autoimmune diseases such as type 1 diabetes (T1D) and multiple sclerosis, where one particular cell type is attacked by the patient's own immune system and needs replacement. Thus, the generation of universal cells that can be safely transplanted into anyone holds the promise of unlocking the full potential of regenerative medicine.

Experimental Procedures
CRISPR gRNA Sequences
HLA-A upstream:
(SEQ ID NO: 1)
5′-GCCGCCTCCCACTTGCGCT-3′
HLA-A downstream:
(SEQ ID NO: 2)
5′-CACATGCAGCCCACGAGCCG-3′
HLA-B upstream_1:
(SEQ ID NO: 3)
5′-ATCCCTAAATATGGTGTCCC-3′
HLA-B upstream_2:
(SEQ ID NO: 4)
5′-TCCCTAAATATGGTGTCCCT-3′
HLA-C downstream_1:
(SEQ ID NO: 5)
5′-GTGATCCGGGTATGGGCAGT-3′
HLA-C downstream_2:
(SEQ ID NO: 6)
5′-TGATCCGGGTATGGGCAGTG-3′
CIITA:
(SEQ ID NO: 7)
5′-TCCATCTGGTCATAGAAG-3′
gRNA_AAVS1-T2:
(SEQ ID NO: 8)
5′-GGGGCCACTAGGGACAGGAT-3′
PCR and qPCR Probes/Primers
PCR primers used in FIG. 6:
Purple_F:
(SEQ ID NO: 9)
5′-CACTCAGAGCAAAGGTCAGATG-3′
Purple_R:
(SEQ ID NO: 10)
5′-AGACTTGAATCCATAAGCCCAA-3′
Red_F:
(SEQ ID NO: 11)
5′-GACAAGTCTCGGAGATGGTTTT-3′
Red_R:
(SEQ ID NO: 12)
5′-AGACTTGAATCCATAAGCCCAA-3′
Green_F:
(SEQ ID NO: 13)
5′-CACTCAGAGCAAAGGTCAGATG-3′
Green_R:
(SEQ ID NO: 14)
5′-TTTGTTGTCAGCCAGACATAGG-3′
Yellow_F:
(SEQ ID NO: 15)
5′-CTGGTTATCTCCCCATTCTCTG-3′
Yellow_R:
(SEQ ID NO: 16)
5′-AAGCATTCACTCCTGACCCTG-3′
Blue_F:
(SEQ ID NO: 17)
5′-GTCTTCCCTCCCAGGCAGCTCA-3′
Blue_R:
(SEQ ID NO: 18)
5′-TGAGGGGTGGGGGATACCGGA-3′
Black_F:
(SEQ ID NO: 19)
5′-TCGACCTACTCTCTTCCGCA-3′
Black_R:
(SEQ ID NO: 20)
5′-TAGGGGGCGTACTTGGCATA-3′
Gray_F:
(SEQ ID NO: 21)
5′-CCGTTCTCCTGTGGATTCGG-3′
Gray_R:
(SEQ ID NO: 22)
5′-TCTCTGGCTCCATCGTAAGC-3′
PCR primers used in FIG. 8:
HLA-F-AS1_F:
(SEQ ID NO: 23)
5′-GTCGCTTCAGTCAGGACACA-3′
HLA-F-AS1_R:
(SEQ ID NO: 24)
5′-GAAGGTGCTGTTTGGCACAG-3′
ITGA6_F:
(SEQ ID NO: 25)
5′-CCTTCAACTTGGACACTCGGG-3′
ITGA6_R:
(SEQ ID NO: 26)
5′-CCACGGGCCAACTACTCC-3′
HEATR1_F:
(SEQ ID NO: 27)
5′-TTACCCAGTTCAATACTGAGCCA-3′
HEATR1_R:
(SEQ ID NO: 28)
5′-AGGGGTAAGCTGCAAACTTCTT-3′
PTDSS2_F:
(SEQ ID NO: 29)
5′-GACCTCCACAGGGACTAGGT-3′
PTDSS2_R:
(SEQ ID NO: 30)
5′-TTTGGAGTTGGTGCTCCCTC-3′
CTBS_F:
(SEQ ID NO: 31)
5′-GCCCTCATCGAGTGGTCAAA-3′
CTBS_R:
(SEQ ID NO: 32)
5′-CCGCTAGACCTGCTGCTATG-3′
ACSBG1_F:
(SEQ ID NO: 33)
5′-CTGGGTGTCAATGATGGCGT-3′
ACSBG1_R:
(SEQ ID NO: 34)
5′-GCCACATCTAAAGGCAGTCG-3′
AC078852.1_F:
(SEQ ID NO: 35)
5′-GTTTGTGGGTGCTGGTCAAC-3′
AC078852.1_R:
(SEQ ID NO: 36)
5′-CTAGGCAACAGTGACAGGGG-3′
HIPK4_F:
(SEQ ID NO: 37)
5′-GGACCATCATGTCGGAGACC-3′
HIPK4_R:
(SEQ ID NO: 38)
5′-GACCTGGGAGTCACACGAAC-3′
ACSBG1_F:
(SEQ ID NO: 39)
5′-CTGGGTGTCAATGATGGCGT-3′
ACSBG1_R:
(SEQ ID NO: 40)
5′-GCCACATCTAAAGGCAGTCG-3′
HIC2_F:
(SEQ ID NO: 41)
5′-AAGTGTTCGGTCTGCGAGAA-3′
HIC2_R:
(SEQ ID NO: 42)
5′-GCTCTGCTTGGTACGGACTG-3′
HLA-H_F:
(SEQ ID NO: 43)
5′-AGGTGATGTATGGCTGCGAC-3′
HLA-H_R:
(SEQ ID NO: 44)
5′-TCCTTCCCGTTCTCCAGGTA-3′
HLA-K_F:
(SEQ ID NO: 45)
5′-GGTATGAACAGCACGCCAAC-3′
HLA-K_R:
(SEQ ID NO: 46)
5′-GCGTCTTGTGTTCCCTGGTA-3′
HLA-G_F:
(SEQ ID NO: 47)
5′-ACCCTCTACCTGGGAGAACC-3′
HLA-G_R:
(SEQ ID NO: 48)
5′-AGGCTCTCCTTTGTTCAGCC-3′
PYCRL_F:
(SEQ ID NO: 49)
5′-CCTAGCCACGTGTGACTCAA-3′
PYCRL_R:
(SEQ ID NO: 50)
5′-TGCCGTCCCAGTAACCAATC-3′
RAB11FIP4_F:
(SEQ ID NO: 51)
5′-CGAGGGAGGGCAAATTGAGT-3′
RAB11FIP4_R:
(SEQ ID NO: 52)
5′-GAAGAAGGGACAAGGGGTGG-3′
CHFR_F:
(SEQ ID NO: 53)
5′-GAGCTTTGATGGCAGAGTGTTA-3′
CHFR_R:
(SEQ ID NO: 54)
5′-CTGGGAGCATGCATTTGTGAGA-3′
PNCK_F:
(SEQ ID NO: 55)
5′-CTGTTGGCAGGTGAACCTCT-3′
PNCK_R:
(SEQ ID NO: 56)
5′-CTGGGAAGGCTTGTCTCCTG-3′
AMN_F:
(SEQ ID NO: 57)
5′-AGAGCTCAAGGTCCCAAGTG-3′
AMN_R:
(SEQ ID NO: 58)
5′-GGGTAACTCACTCGGAGGTC-3′
FUT1_F:
(SEQ ID NO: 59)
5′-TGGATTTCCAGAACCCCATCC-3′
FUT1_R:
(SEQ ID NO: 60)
5′-GGGAACTCTCCCTCTGGTCT-3′
NPPA_F:
(SEQ ID NO: 61)
5′-GAGCTTCTGCATTGGTCCCT-3′
NPPA_R:
(SEQ ID NO: 62)
5′-TCTGATCGATCTGCCCTCCT-3′
SYBR-based qPCR primers:
AFP_F:
(SEQ ID NO: 63)
5′-AAATGCGTTTCTCGTTGCTT-3′
AFP_R:
(SEQ ID NO: 64)
5′-GCCACAGGCCAATAGTTTGT-3′
SOX17_F:
(SEQ ID NO: 65)
5′-CTCTGCCTCCTCCACGAA-3′
SOX17_R:
(SEQ ID NO: 66)
5′-CAGAATCCAGACCTGCACAA-3′
BRACHYURY_F:
(SEQ ID NO: 67)
5′-AATTGGTCCAGCCTTGGAAT-3′
BRACHYURY_R:
(SEQ ID NO: 68)
5′-CGTTGCTCACAGACCACA-3′
FLK1_F:
(SEQ ID NO: 69)
5′-TGATCGGAAATGACACTGGA-3′
FLK1_R:
(SEQ ID NO: 70)
5′-CACGACTCCATGTTGGTCAC-3′
MAP2_F:
(SEQ ID NO: 71)
5′-CAGGTGGCGGACGTGTGAAAATTGAGAGTG-3′
MAP2_R:
(SEQ ID NO: 72)
5′-CACGCTGGATCTGCCTGGGGACTGTG-3′
PAX6_F:
(SEQ ID NO: 73)
5′-GTCCATCTTTGCTTGGGAAA-3′
PAX6_R:
(SEQ ID NO: 74)
5′-TAGCCAGGTTGCGAAGAACT-3′

### TaqMan Gene Expression Assays:

### HLA-E: Hs03045171_m1

### CD8: Hs00233520_m1

### IL-2: Hs00174114_m1

RPLP0 (internal control): Hs99999902_m1

### FACS Antibodies

### α-HLA-A2 (PE-conjugated), Clone BB7.2, Biolegend, Cat #343305

### α-HLA-ABC (PE-conjugated), Clone W6/32, Biolegend, Cat #311406

### α-HLA-E (PE-conjugated), Clone 3D12, Biolegend, Cat #342603

α-HLA-G (PE-conjugated), Clone MEM-G/9, Abcam, Cat #ab24384

### α-HLA-DR (APC-conjugated), Clone MEM-12, ThermoFisher Scientific, Cat #MA1-10347

### α-B2M (APC-conjugated), Clone 2M2, Biolegend Cat #316311

### α-PD-L1 (APC-conjugated), Clone 29E.2A3, Biolegend, Cat #329708

### α-PD-1 (APC-conjugated), Clone EH12.2H7, Biolegend, Cat #329908

### α-CD3 (APC-conjugated), Clone UCHT1, Biolegend, Cat #300412

α-CD3 (Pacific Blue™-conjugated), Clone UCHT1, Biolegend, Cat #300418
α-CD4 (PE/Cy7-conjugated), Clone RPA-T4, Biolegend, Cat #300511

### α-CD8 (PE-conjugated), Clone SK1, Biolegend, Cat #344705

α-CD25 (Alexa Fluor® 700-conjugated), Clone M-A251, Biolegend, Cat #356117

### α-CD47 (PE-conjugated), Clone CC2C6, Biolegend, Cat #323108

### α-CD56 (PE-conjugated), Clone HCD56, Biolegend, Cat #318306

α-CD69 (Alexa Fluor® 647-conjugated), Clone FN50, Biolegend, Cat #310918

### α-CD107a (APC-conjugated), Clone H4A3, Biolegend, Cat #328620

### α-CD144 (PE-conjugated), Clone 55-7H1, BD Biosciences, Cat #560410

### Isotypes

### Isotype 1: Mouse IgG2b, κ Isotype Control (APC-conjugated), Biolegend, Cat #400322

### Isotype 2: Mouse IgG2b, κ Isotype Control (PE-conjugated), Biolegend, Cat #401208

### Isotype 3: Mouse IgG2a, κ Isotype Control (PE-conjugated), Biolegend, Cat #400214

### Immunofluorescence Antibodies

α-OCT4, Abcam, Cat #ab19857
α-NANOG, Abcam Cat #ab21624

### α-SSEA3, Millipore, Cat #MAB4303

### α-SSEA4, Millipore, Cat #MAB4304

### α-TRA-1-60, Millipore, Cat #MAB4360

Donkey anti-Rabbit IgG (H+L) Secondary Antibody, Alexa Fluor® 488 conjugate, Life Technologies, Cat #A-21206
Donkey anti-Mouse IgG (H+L) Secondary Antibody, Alexa Fluor® 488 conjugate, Life Technologies, Cat #A-21202
Goat anti-Mouse IgM Heavy Chain Secondary Antibody, Alexa Fluor® 555 conjugate, Life Technologies, Cat #A-21426

### Human ES Cell Culture, Electroporation, and Drug Selection

HUES8 cells (Cowan et al., 2004) were grown on Geltrex (Life Technologies) pre-coated plates and cultured in mTeSR1 (StemCell Technologies) supplemented with penicillin/streptomycin. For passaging, cells were dissociated with Gentle Cell Dissociation Reagent (StemCell Technologies) for 5-10 min and replated in fresh media supplemented with RevitaCell™ (ThermoFisher Scientific). For electroporation, as previously described (Peters et al., 2013), HUES8 cells were dissociated into singles cells and 10 million cells were electroporated with 50 μg of pCas9_GFP (Addgene #44719) and a total of 50 μg of gRNA plasmid for gene knockout. For gene knock-in into the AAVS1 locus, cells were electroporated with 50 μg of pCas9_GFP, 25 μg of gRNA_AAVS1-T2 (Addgene #41818), and 40 μg of double-stranded donor plasmid. For gene knock-out purpose, the cells were collected 48 hrs post-electroporation. GFP-expressing cells were enriched by FACS (FACSAria II, BD Biosciences) and replated on 10 cm tissue-culture plates at 15,000 cells/plate in fresh media supplemented with RevitaCell™, to allow single cell colony formation. Alternatively, for gene knock-in, 48 hrs post-electroporation cells were selected by blasticidin (ThermoFisher Scientific) at 2 μg/ml for 5 days. Cell colonies were then manually picked and expanded.

### CRISPR/Cas9 Genome Editing

Five hundred base pairs of each region upstream or downstream of HLA-A/B/C were amplified from HUES8 or HEK293T cells and Sanger-sequenced (Genewiz). The sequence conserved between the two cell lines was chosen as reference sequence, and sgRNAs were designed using the CRISPR design tool developed by Feng Zhang's lab at MIT (available at: crispr.mit.edu) and CCTop (Stemmer et al., 2015). Top ranked sgRNAs were picked and cloned into a gRNA expression vector (Addgene #41824). The gRNA plasmid was then transfected into HEK293T cells, genomic DNA was extracted and PCR amplicons covering the cutting site were analyzed by TIDE (available at: tide.nki.nl) for on-target efficiency. Single guide RNAs with the highest on-target activities were used for genome editing in HUES8 cells. To build the knock-in donor plasmid, the ORFs of PD-L1, HLA-G, and CD47 were individually cloned and connected by 2A sequence using Gibson Assembly® (New England BioLabs). The 3-in-1 cassette was then inserted into the AAVS1-Blasticidine-CAG-Flpe-ERT2 plasmid (Addgene #68461) between Sal I and Mlu I restriction sites, after Flpe-ERT2 was cut out. Details on genome editing of human ESCs were previously described (Peters et al., 2013).

### Generation of HLA Knockout (KO) Cell Line

Briefly, to knockout the adjacent HLA-B/-C genes, a total of four sgRNAs was co-electroporated together with a Cas9 expression plasmid into wild-type (WT) HUES8. Primers shown in FIG. 6A were then used to screen for homozygous knockout clones (HLA-B/-C^{-/-} efficiency: 1.56%). Heterozygous knockout clones were also observed (HLA-B/-C^{+/-} efficiency: 7.8%). The homozygous clones were further verified for ablation of HLA-B/-C mRNA expression by RT-PCR and normal karyotypes were confirmed by nCounter Human Karyotype Assay (data not shown). At last, one karyotypically normal clone was chosen for further targeting of the HLA-A and CITTA genes in one electroporation. PCR with the primers shown in FIG. 6B and flow cytometry using an α-HLA-A2 antibody were carried out to screen for HLA-A knockout clones. Primers shown in FIG. 6E and Sanger sequencing were performed to identify CIITA knockout clones. As a result, only heterozygous clones (HLA-A^{+/-}CIITA^{+/indel}) were observed after the first round of HLAA/CIITA targeting (HLA-A^{+/-} efficiency: 3.68%). Therefore, another round of electroporation with HLA-A/CIITA sgRNAs was applied to one karyotypically normal heterozygous clone, and the same screening strategies were employed. At last, one homozygous clone (HLA-A^{-/-} CIITA^{indel/indel}) was generated, however, FACS analysis revealed that this clone was an admixed clone, which still retained 1% HLA-A⁺ cells. After subcloning, a pure homozygous clone (HLA Knockout, KO) was obtained.

### Karyotyping

Karyotype G-banding was performed by Cell Line Genetics.

Directed Differentiation into Three Germ Layers

WT and gene-edited HuES8 cell lines were differentiated into ectoderm, mesoderm, and endoderm following the monolayer-based protocols of the STEMdiff™ Trilineage Differentiation Kit (StemCell Technologies).

Differentiation into Endothelial Cells and Vascular Smooth Muscle Cells

Human endothelial cells (EC) and vascular smooth muscle cells (VSMC) were differentiated following the published protocols (Patsch et al., 2015). Briefly, for EC differentiation, ESCs were plated in N2B27 media supplemented with 8 uM CHIR99021 (Cayman Chemical) and 25 ng/ml BMP4 (Peprotech) for 3 days to induce lateral mesoderm. Media were then replaced with StemPro-34 supplemented with 200 ng/ml VEGF (Peprotech) and 2 μM forskolin (Abcam) for 2 days to induce EC. Cells were then enriched for CD144⁺ cells using MACS cell separation (Miltenyi Biotec). The CD144⁺ cells were plated on Fibronectin (Corning)-coated plates in EBM™-2 supplemented with EGM™-2 BulletKit™ (Lonza) for further differentiation for at least 7 days. For VSMC differentiation, ESCs were plated in the same media for 3 days as for EC differentiation. On day 4 and 5, media were changed to N2B27 supplemented with 12.5 ng/ml PDGF-BB (Peprotech) and 12.5 ng/ml Activin A (Cell Guidance Systems). From day 6 onwards, cells were dissociated and plated on gelatin-coated dishes in Medium 231 supplemented with Smooth Muscle Growth Supplement (ThermoFisher Scientific) for further differentiation.

### Human Primary Immune Cell Isolation and Culture

Blood was obtained from healthy, de-identified donors (leukopaks) from the Jackson Transfusion Center at Massachusetts General Hospital, Boston. Human primary T cells, NK cells, or CD14⁺ monocytes were isolated by negative selection kits (RosetteSep™ Human T Cell Enrichment Cocktail, RosetteSep™ Human NK Cell Enrichment Cocktail, and RosetteSep™ Human Monocyte Enrichment Cocktail, StemCell Technologies), respectively. Isolated T cells were cultured in X-VIVO 10 (Lonza) media supplemented with 5% Human AB Serum (Valley Biomedical), 5% Fetal Bovine Serum, 1% Penicillin/Streptomycin, GlutaMAX, MEM Non-Essential Amino Acids (ThermoFisher Scientific), and 20 U/ml IL-2 (Peprotech). Isolated NK cells were cultured in RPMI 1640 with L-Glutamine (Corning) supplemented with 10% Fetal Bovine Serum and 1% Penicillin/Streptomycin. Isolated monocytes were differentiated into macrophages in RPMI 1640 supplemented with 10% Fetal Bovine Serum, 1% Penicillin/Streptomycin, and 25-50 ng/ml M-CSF (Peprotech).

### Flow Cytometry

PBS containing 1% Fetal Bovine Serum (FBS) was used as washing and staining buffer; PBS containing 4% FBS was used as blocking buffer. In the case of ECs and VSMCs, FcR blocking reagent (Miltenyi Biotec) was added to the blocking buffer at a 1:1000 dilution. Briefly, immune cells or other dissociated single cells were washed once and blocked with blocking buffer on ice for 20 min. Cells were stained with antibodies on ice for 30-60 min and washed twice before analysis on a FACSCalibur™ or LSR II (BD Biosciences). The data were plotted using FlowJo software (BD).

### In Vitro T Cell Proliferation Assay

When VSMCs were used, cells were first treated with mitomycin (Fisher Scientific). One hundred thousand ECs or VSMCs were plated on 24-well plates and treated with IFNγ (100 ng/ml) for 48 hrs before the assay. On day 0 of co-incubation, isolated CD3⁺ T cells were labeled with CellTrace™ CFSE (ThermoFisher Scientific) following the manufacturer's instructions. Adherent ECs or VSMCs were washed twice with PBS before co-incubation with 500 k CFSE-labeled T cells in T cell culture media supplemented with 20 U/ml IL-2 for 5 days. T cells were then stained with anti-CD3/4/8 antibodies before being analyzed on an LSR II for CFSE intensity. T cells cultured for 5 days without target cells were used as negative control. T cells treated with Dynabeads™ Human T-Activator CD3/CD28 beads (ThermoFisher Scientific) for 5 days served as positive control.

### In Vitro T Cell Activation Assay and Cytokine Secretion Assay

ESC-derived ECs were used as target cells. The conditions for co-culture were the same as in the T cell proliferation assay, except that the T cells were not labeled. After 5-day-co-culture, T cells were stained for T cell activation markers before being analyzed on an LSR II. For multiple secreted cytokine quantifications, supernatants were collected and analyzed by customized MSD U-PLEX Platform (Meso Scale Discovery) following manufacturer's instructions. T cells or target cells cultured for 5 days were used as negative control. T cells activated with Dynabeads™ Human T-Activator CD3/CD28 beads (ThermoFisher Scientific) for 5 days served as positive control. Background activation was assessed using T cells incubated with conditioned media from ECs or VSMCs. Conditioned media was prepared as described above.

### In Vitro T/NK Cell Killing Assay

ESC-derived VSMCs were used as target cells. For T cell killing assay, the conditions for co-incubation were the same as in the T cell activation assay. For NK cell killing assay, 40K VSMCs and NK cells at the indicated effector/target ratios were co-incubated in 200 μl NK cell medium in 96-well U bottom for 20 hrs before the supernatants were harvested. After co-incubation, supernatants were collected and analyzed by Pierce™ LDH Cytotoxicity Assay Kit (ThermoFisher Scientific) following the manufacturer's instructions. T cell medium or NK cell medium (RPMI-10) was used as background control. T/NK cells cultured alone or target cells cultured alone were used as controls for spontaneous LDH release. Lysed target cells at endpoint were used as maximum LDH release.

### Pre-sensitization of Allogeneic Human CD8+ T Cells

Human primary CD8⁺ T cells were isolated using RosetteSep™ Human CD8⁺ T Cell Enrichment Cocktail (StemCell Technologies), and pre-sensitized with HUES8-derived embryoid bodies as previously described (Gornalusse et al., 2017). Briefly, the embryoid bodies were induced in suspension for 5 days followed by attachment culture for another 4 days. CD8⁺ T cells were then co-cultured with attached embryoid body cells for pre-sensitization. Extracellular matrix from xenogeneic resources such as Gelatin was avoided during this process to prevent unspecific T cell activation.

### In Vivo T Cell Recall Response Assay

All animal experiments were performed in accordance to Harvard University International Animal Care and Use Committee regulations. No randomization was used. All procedures were done in a blinded fashion. Male immunodeficient SCID Beige mice (Taconic) aged 8-10 weeks were used for teratoma formation. Two million HUES8 cells were encapsulated in a blood clot, and the blood clot was inserted subcutaneously into each flank of the SCID Beige mice. Teratoma size was measured by caliper weekly after the teratoma became palpable. Four to six weeks after hESC transplantation, one million pre-sensitized allogeneic human CD8⁺ T cells were injected via tail vein into the mice. Following T cell injection, teratoma size was measured on day 2, day 5, and day 7; teratoma size was also measured 2 days before the T cell injection. On day 8 post-injection, the teratoma were harvested and analyzed by qPCR and hematoxylin and eosin (H&E) staining Two allogeneic CD8⁺ T cell donors were used in the same experimental condition and the results were combined in this study. Histology was performed by the histology core of the Harvard Stem Cell Institute.

### In Vitro NK Cell Degranulation Assay

Three hundred thousand adherent ESC-derived VSMCs were seeded in 24-well plates 24 hrs before the assay. The next day, VSMCs were washed once with PBS before co-incubation with 100K freshly isolated NK cells in NK cell media supplemented with α-CD107a APC (Biolegend) and eBioscience™ Protein Transport Inhibitor Cocktail (ThermoFisher Scientific). After NK cells were added into the wells, the plate was spun down at 2,000 rpm for 5 min to achieve sufficient effector-target contact. After a 20 h-co-incubation the NK cells were stained with α-CD56 PE (Biolegend) before analysis on a FACSCalibur™ for CD107a cell surface expression. NK cell cultures without target cells were used as negative control. NK cells treated with Cell Activation Cocktail (without Brefeldin A), which includes PMA (phorbol 12-myristate-13-acetate) and ionomycin, were used as positive control for degranulation.

### In Vitro Macrophage Phagocytosis Assay

Monocytes were isolated from donor blood via negative selection using RosetteSep™ Human Monocyte Enrichment Cocktail (StemCell Technologies). Monocytes were plated in serum-free medium for adhesion and maturation into macrophages for one to three weeks in RPMI 1640 supplemented with 10% FBS, 1% Penicillin/Streptomycin, and 25 ng/ml of M-CSF (Peprotech). Macrophages were replated in 96-well μ-plates (ibidi) at a density of 100K/well two days before the assay. For the assay, differentiated VSMCs were pretreated with 200 nM staurosporine (Sigma) for 1.5 hrs to be used for the “STS treated” group. VSMCs were dissociated and labeled with pHrodo-Red (IncuCyte) for 1 h in 37° C. Thirty thousand labeled VSMCs were added into each well containing macrophages, and the co-incubated culture was immediately transferred into the Celldiscover 7 live cell imaging platform (Zeiss). One image per well of the red fluorescence emission upon phagocytic engulfment was acquired every 20 min for 6 hrs. Total integrated intensity (mean fluorescence intensity*total area) was analyzed for each image using the ZEN imaging software (Zeiss). The pHrodo-Red⁺ particles indicate phagosomes within the macrophages that have engulfed VSMCs.

### Generation of CD47−/− and B2M−/− HUES8 Cell Lines

The following four CRISPR sgRNAs were used to target the first coding exon of CD47 in HUES8 cells.

(SEQ ID NO: 75)
5′-gGTCCTGCCTGTAACGGCGG-3′
(SEQ ID NO: 76)
5′-gGACCGCCGCCGCGCGTCAC-3′
(SEQ ID NO: 77)
5′-gCAGCAACAGCGCCGCTACC-3′
(SEQ ID NO: 78)
5′-gTTCGCCCCCGCGGGCGTGT-3′

Cells were stained 72 hrs post electroporation with an anti-CD47 antibody (Clone CC2C6), and CD47 negative cells were isolated using a FACS Aria (BD). Single cell-derived colonies were obtained as described previously (Peters et al., 2013), and subsequently loss of CD47 expression was confirmed by FACS analysis. Similarly, a Beta-2-Microglubulin (B2M)-deficient HUES8 cell line was generated using the following sgRNA:

(SEQ ID NO: 79)
5′-gCTACTCTCTCTTTCTGGCC-3′.

### Lentiviral Transduction

A doxycycline-inducible lentiviral Gateway vector (Invitrogen) containing the PD-L1 ORF was constructed by PCR amplification. PD-L1 expression lentiviruses were packaged by transfecting HEK293T cells with the PD-L1-expressing vector and the packaging plasmids pMDL, pVSVG, and pREV. Medium containing lentiviral particles was collected 48 hr post-transfection and used to transduce VSMCs along with lentiviral particles encoding the doxycycline-binding transactivator rtTA. After 24 hrs, VSMCs were treated with doxycycline (10 μg/ml) to induce PD-L1 expression that was verified by FACS. Assessment of T cell proliferation against VSMCs overexpressing PD-L1 was performed as described above, except for a 7-day-co-incubation and the presence of doxycycline throughout the co-incubation. No effect on T cell proliferation was observed by the addition of doxycycline.

### Immunofluorescence

PBS containing 0.05% Tween-20 was the washing buffer between each step after cells were fixed. Briefly, cells were washed with PBS, fixed with 4% paraformaldehyde, and permeablized with 0.1% Triton X-100. Cells were blocked with 4% Donkey Serum (Jackson ImmunoResearch Laboratories) at 4° C. overnight and incubated with appropriate primary antibodies diluted in blocking buffer at RT for 1 hr. Cells were then incubated with Alexa Fluor® 488- or Alexa Fluor® 555-conjugated secondary antibodies (Life Technologies). Cells were washed and nuclei were stained with Hoechst. Images were visualized with a Nikon inverted microscope.

RNA Isolation, cDNA Synthesis and qPCR

RNA was extracted using TRIzol Reagent (ThermoFisher Scientific) according to the manufacturer's instructions. cDNA synthesis was done using SuperScript VILO cDNA synthesis kit (ThermoFisher Scientific) according to the manufacturer's protocol. SYBR green-based or TaqMan-based qPCR was performed, and relative quantification was determined using the QuantStudio 12 k Flex System (ThermoFisher Scientific) and then calculated by means of the comparative Ct method (2^{-ΔΔQ}) relative to the expression of the respective internal control.

### Next Generation Sequencing (NGS)-Based Off-Target Analysis

The off-target sites were predicted using CCTop (Stemmer et al., 2015). The bait design, the enrichment of genomic DNA (library preparation), and NGS were conducted by Arbor Biosciences using myBaits® custom target capture kit. Briefly, for each of the 648 predicted off-target sites, five RNA baits were designed across each off-target site and placed every ˜26 bp, covering a 181-182 bp window. Following genomic DNA extraction from WT as well as from the three engineered hPSC lines, the biotinylated RNA baits were hybridized to the corresponding denatured genomic DNA library. Subsequently, the RNA-gDNA hybrids were bound to streptavidin-coated beads and non-specific bonds were washed off. The remaining gDNA libraries were amplified and sequenced by paired-end NGS using NovaSeq (Illumina).

Genome editing events were quantified by CRISPRessoPooled from CRISPResso suite (Version 1.0.13) with default settings unless stated later (Pinello et al., 2016). In brief, for each of the four libraries, the reads with minimum single base pair score (phred33) greater than 25 were selected and aligned to a ±100 bp window around each gRNA off-target site in the human genome (hg38). The sites (=3) with fewer than 5 aligned reads in any of the libraries were filtered out. The percentage of reads with altered sequences (insertion, deletion, and substitution) compared to hg38 at each off-target site from each library was calculated by the program. If the % reads with altered sequence was found >0 in WT as well as in all three engineered lines, the sequences were further inspected. In case the sequences of all three engineered lines matched the WT sequence, they were classified as SNP/PM; however, in case the sequences from the engineered cell lines deviated from the WT sequence, they were identified as editing events. Polymorphisms (PM) represent small deletions/insertions instead of single nucleotide polymorphisms (SNPs) observed already in the WT hPSCs, deviating from hg38.

### Statistical Analyses

Plots were generated, and statistical analyses were performed using Prism 7 (Graphpad).

### REFERENCES

Abrahimi, P., Chang, W. G., Kluger, M. S., Qyang, Y., Tellides, G., Saltzman, W. M., and Pober, J. S. (2015). Efficient gene disruption in cultured primary human endothelial cells by CRISPR/Cas9. Circ Res 117, 121-128.
Baldwin, W. M., 3rd, Valujskikh, A., and Fairchild, R. L. (2016). Mechanisms of antibody-mediated acute and chronic rejection of kidney allografts. Curr Opin Organ Transplant 21, 7-14.
Beldi-Ferchiou, A., Lambert, M., Dogniaux, S., Vely, F., Vivier, E., Olive, D., Dupuy, S., Levasseur, F., Zucman, D., Lebbe, C., et al. (2016). PD-1 mediates functional exhaustion of activated NK cells in patients with Kaposi sarcoma. Oncotarget 7, 72961-72977.
Betancur, P. A., Abraham, B. J., Yiu, Y. Y., Willingham, S. B., Khameneh, F., Zarnegar, M., Kuo, A. H., McKenna, K., Kojima, Y., Leeper, N. J., et al. (2017). A CD47-associated super-enhancer links pro-inflammatory signalling to CD47 upregulation in breast cancer. Nat Commun 8, 14802.
Bour-Jordan, H., Salomon, B. L., Thompson, H. L., Szot, G. L., Bernhard, M. R., and Bluestone, J. A. (2004). Costimulation controls diabetes by altering the balance of pathogenic and regulatory T cells. J Clin Invest 114, 979-987.
Braud, V. M., Allan, D. S., O'Callaghan, C. A., Soderstrom, K., D'Andrea, A., Ogg, G. S., Lazetic, S., Young, N. T., Bell, J. I., Phillips, J. H., et al. (1998a). HLA-E binds to natural killer cell receptors CD94/NKG2A, B and C. Nature 391, 795-799.
Braud, V. M., Allan, D. S., Wilson, D., and McMichael, A. J. (1998b). TAP- and tapasin-dependent HLA-E surface expression correlates with the binding of an MHC class I leader peptide. Curr Biol 8, 1-10.
Brehm, M. A., Wiles, M. V., Greiner, D. L., and Shultz, L. D. (2014). Generation of improved humanized mouse models for human infectious diseases J Immunol Methods 410, 3-17.
Chen, H., Li, Y., Lin, X., Cui, D., Cui, C., Li, H., and Xiao, L. (2015). Functional disruption of human leukocyte antigen II in human embryonic stem cell. Biol Res 48, 59.
Chhabra, A., Ring, A. M., Weiskopf, K., Schnorr, P. J., Gordon, S., Le, A. C., Kwon, H. S., Ring, N. G., Volkmer, J., Ho, P. Y., et al. (2016). Hematopoietic stem cell transplantation in immunocompetent hosts without radiation or chemotherapy. Sci Transl Med 8, 351ra105.
Collins, T., Korman, A. J., Wake, C. T., Boss, J. M., Kappes, D. J., Fiers, W., Ault, K. A., Gimbrone, M. A., Jr., Strominger, J. L., and Pober, J. S. (1984) Immune interferon activates multiple class II major histocompatibility complex genes and the associated invariant chain gene in human endothelial cells and dermal fibroblasts. Proc Natl Acad Sci USA 81, 4917-4921.
Cowan, C. A., Klimanskaya, I., McMahon, J., Atienza, J., Witmyer, J., Zucker, J. P., Wang, S., Morton, C. C., McMahon, A. P., Powers, D., et al. (2004). Derivation of embryonic stem-cell lines from human blastocysts. N Engl J Med 350, 1353-1356.
de Almeida, P. E., Ransohoff, J. D., Nahid, A., and Wu, J. C. (2013). Immunogenicity of pluripotent stem cells and their derivatives. Circ Res 112, 549-561.
de Rham, C., and Villard, J. (2014). Potential and limitation of HLA-based banking of human pluripotent stem cells for cell therapy J Immunol Res 2014, 518135.
Della Chiesa, M., Pesce, S., Muccio, L., Carlomagno, S., Sivori, S., Moretta, A., and Marcenaro, E. (2016). Features of Memory-Like and PD-1(+) Human NK Cell Subsets. Front Immunol 7, 351.
Diehl, M., Munz, C., Keilholz, W., Stevanovic, S., Holmes, N., Loke, Y. W., and Rammensee, H. G. (1996). Nonclassical HLA-G molecules are classical peptide presenters. Curr Biol 6, 305-314.
Ding, Q., Regan, S. N., Xia, Y., Oostrom, L. A., Cowan, C. A., and Musunuru, K. (2013) Enhanced efficiency of human pluripotent stem cell genome editing through replacing TALENs with CRISPRs. Cell Stem Cell 12, 393-394.
Djamali, A., Kaufman, D. B., Ellis, T. M., Zhong, W., Matas, A., and Samaniego, M. (2014). Diagnosis and management of antibody-mediated rejection: current status and novel approaches. Am J Transplant 14, 255-271.
Drukker, M., Katz, G., Urbach, A., Schuldiner, M., Markel, G., Itskovitz-Eldor, J., Reubinoff, B., Mandelboim, O., and Benvenisty, N. (2002). Characterization of the expression of MHC proteins in human embryonic stem cells. Proc Natl Acad Sci USA 99, 9864-9869.
Feder, J. N., Gnirke, A., Thomas, W., Tsuchihashi, Z., Ruddy, D. A., Basava, A., Dormishian, F., Domingo, R., Jr., Ellis, M. C., Fullan, A., et al. (1996). A novel MHC class I-like gene is mutated in patients with hereditary haemochromatosis. Nat Genet 13, 399-408.
Ferreira, L. M. R., Meissner, T. B., Tilburgs, T., and Strominger, J. L. (2017). HLA-G: At the Interface of Maternal-Fetal Tolerance. Trends Immunol 38, 272-286.
Glas, R., Franksson, L., Ohlen, C., Hoglund, P., Koller, B., Ljunggren, H. G., and Karre, K. (1992). Major histocompatibility complex class I-specific and -restricted killing of beta 2-microglobulin-deficient cells by CD8⁺ cytotoxic T lymphocytes. Proc Natl Acad Sci USA 89, 11381-11385.
Gordon, S. R., Maute, R. L., Dulken, B. W., Hutter, G., George, B. M.,
McCracken, M. N., Gupta, R., Tsai, J. M., Sinha, R., Corey, D., et al. (2017). PD-1 expression by tumour-associated macrophages inhibits phagocytosis and tumour immunity. Nature 545, 495-499.
Gornalusse, G. G., Hirata, R. K., Funk, S. E., Riolobos, L., Lopes, V. S., Manske, G., Prunkard, D., Colunga, A. G., Hanafi, L. A., Clegg, D. O., et al. (2017). HLA-E-expressing pluripotent stem cells escape allogeneic responses and lysis by NK cells. Nat Biotechnol 35, 765-772.
Iniotaki-Theodoraki, A. (2001). The role of HLA class I and class II antibodies in renal transplantation. Nephrol Dial Transplant 16 Suppl 6, 150-152.
Jaiswal, S., Jamieson, C. H., Pang, W. W., Park, C. Y., Chao, M. P., Majeti, R., Traver, D., van Rooijen, N., and Weissman, I. L. (2009). CD47 is upregulated on circulating hematopoietic stem cells and leukemia cells to avoid phagocytosis. Cell 138, 271-285.
Krawczyk, M., and Reith, W. (2006). Regulation of MHC class II expression, a unique regulatory system identified by the study of a primary immunodeficiency disease. Tissue Antigens 67, 183-197.
Lee, N., Goodlett, D. R., Ishitani, A., Marquardt, H., and Geraghty, D. E. (1998a). HLA-E surface expression depends on binding of TAP-dependent peptides derived from certain HLA class I signal sequences. J Immunol 160, 4951-4960.
Lee, N., Llano, M., Carretero, M., Ishitani, A., Navarro, F., Lopez-Botet, M., and Geraghty, D. E. (1998b). HLA-E is a major ligand for the natural killer inhibitory receptor CD94/NKG2A. Proc Natl Acad Sci USA 95, 5199-5204.
Li, Y., Masse-Ranson, G., Garcia, Z., Bruel, T., Kok, A., Strick-Marchand, H., Jouvion, G., Serafini, N., Lim, A. I., Dusseaux, M., et al. (2018). A human immune system mouse model with robust lymph node development. Nat Methods 15, 623-630.
Majeti, R., Chao, M. P., Alizadeh, A. A., Pang, W. W., Jaiswal, S., Gibbs, K. D., Jr., van Rooijen, N., and Weissman, I. L. (2009). CD47 is an adverse prognostic factor and therapeutic antibody target on human acute myeloid leukemia stem cells. Cell 138, 286-299.
Mandal, P. K., Ferreira, L. M., Collins, R., Meissner, T. B., Boutwell, C. L., Friesen, M., Vrbanac, V., Garrison, B. S., Stortchevoi, A., Bryder, D., et al. (2014). Efficient ablation of genes in human hematopoietic stem and effector cells using CRISPR/Cas9. Cell Stem Cell 15, 643-652.
Masson, E., Stern, M., Chabod, J., Thevenin, C., Gonin, F., Rebibou, J. M., and Tiberghien, P. (2007). Hyperacute rejection after lung transplantation caused by undetected low-titer anti-HLA antibodies. J Heart Lung Transplant 26, 642-645.
Mattapally, S., Pawlik, K. M., Fast, V. G., Zumaquero, E., Lund, F. E., Randall, T. D., Townes, T. M., and Zhang, J. (2018). Human Leukocyte Antigen Class I and II Knockout Human Induced Pluripotent Stem Cell-Derived Cells: Universal Donor for Cell Therapy. J Am Heart Assoc 7, e010239.
Meissner, T. B., Mandal, P. K., Ferreira, L. M., Rossi, D. J., and Cowan, C. A. (2014). Genome editing for human gene therapy. Methods Enzymol 546, 273-295.
Melkus, M. W., Estes, J. D., Padgett-Thomas, A., Gatlin, J., Denton, P. W., Othieno, F. A., Wege, A. K., Haase, A. T., and Garcia, J. V. (2006). Humanized mice mount specific adaptive and innate immune responses to EBV and TSST-1. Nat Med 12, 1316-1322.
Meri, S., Morgan, B. P., Davies, A., Daniels, R. H., Olavesen, M. G., Waldmann, H., and Lachmann, P J (1990). Human protectin (CD59), an 18,000-20,000 MW complement lysis restricting factor, inhibits C5b-8 catalysed insertion of C9 into lipid bilayers. Immunology 71, 1-9.
Michaels, P. J., Fishbein, M. C., and Colvin, R. B. (2003). Humoral rejection of human organ transplants. Springer Semin Immunopathol 25, 119-140.
Nakajima, F., Tokunaga, K., and Nakatsuji, N. (2007). Human leukocyte antigen matching estimations in a hypothetical bank of human embryonic stem cell lines in the Japanese population for use in cell transplantation therapy. Stem Cells 25, 983-985.
Patsch, C., Challet-Meylan, L., Thoma, E. C., Urich, E., Heckel, T., O'Sullivan, J. F., Grainger, S. J., Kapp, F. G., Sun, L., Christensen, K., et al. (2015). Generation of vascular endothelial and smooth muscle cells from human pluripotent stem cells. Nat Cell Biol 17, 994-1003.
Pazmany, L., Mandelboim, O., Vales-Gomez, M., Davis, D. M., Reyburn, H. T., and Strominger, J. L. (1996). Protection from natural killer cell-mediated lysis by HLA-G expression on target cells. Science 274, 792-795.
Pegram, H. J., Andrews, D. M., Smyth, M. J., Darcy, P. K., and Kershaw, M. H. (2011). Activating and inhibitory receptors of natural killer cells Immunol Cell Biol 89, 216-224.
Peters, D. T., Cowan, C. A., and Musunuru, K. (2013). Genome editing in human pluripotent stem cells. In StemBook (Cambridge (Mass.)).
Pinello, L., Canver, M. C., Hoban, M. D., Orkin, S. H., Kohn, D. B., Bauer, D. E., and Yuan, G. C. (2016). Analyzing CRISPR genome-editing experiments with CRISPResso. Nat Biotechnol 34, 695-697.
Raulet, D. H. (2006). Missing self recognition and self tolerance of natural killer (NK) cells. Semin Immunol 18, 145-150.
Reith, W., and Mach, B. (2001). The bare lymphocyte syndrome and the regulation of MHC expression. Annu Rev Immunol 19, 331-373.
Riley, J. L. (2009). PD-1 signaling in primary T cells Immunol Rev 229, 114-125.
Riolobos, L., Hirata, R. K., Turtle, C. J., Wang, P. R., Gornalusse, G. G., Zavajlevski, M., Riddell, S. R., and Russell, D. W. (2013). HLA engineering of human pluripotent stem cells. Mol Ther 21, 1232-1241.
Rong, Z., Wang, M., Hu, Z., Stradner, M., Zhu, S., Kong, H., Yi, H., Goldrath, A., Yang, Y. G., Xu, Y., et al. (2014). An effective approach to prevent immune rejection of human ESC-derived allografts. Cell Stem Cell 14, 121-130.
Rongvaux, A., Willinger, T., Martinek, J., Strowig, T., Gearty, S. V., Teichmann, L L, Saito, Y., Marches, F., Halene, S., Palucka, A. K., et al. (2014). Development and function of human innate immune cells in a humanized mouse model. Nat Biotechnol 32, 364-372.
Roth, T. L., Puig-Saus, C., Yu, R., Shifrut, E., Carnevale, J., Li, P. J., Hiatt, J., Saco, J., Krystofinski, P., Li, H., et al. (2018). Reprogramming human T cell function and specificity with non-viral genome targeting. Nature 559, 405-409.
Sadelain, M., Papapetrou, E. P., and Bushman, F. D. (2011). Safe harbours for the integration of new DNA in the human genome. Nat Rev Cancer 12, 51-58.
Salomon, B., and Bluestone, J. A. (2001). Complexities of CD28/B7: CTLA-4 costimulatory pathways in autoimmunity and transplantation. Annu Rev Immunol 19, 225-252.
Stemmer, M., Thumberger, T., Del Sol Keyer, M., Wittbrodt, J., and Mateo, J. L. (2015). CCTop: An Intuitive, Flexible and Reliable CRISPR/Cas9 Target Prediction Tool. PLoS One 10, e0124633.
Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
Tapia, N., and Scholer, H. R. (2016). Molecular Obstacles to Clinical Translation of iPSCs. Cell Stem Cell 19, 298-309.
Taylor, C. J., Bolton, E. M., Pocock, S., Sharples, L. D., Pedersen, R. A., and Bradley, J. A. (2005). Banking on human embryonic stem cells: estimating the number of donor cell lines needed for HLA matching. Lancet 366, 2019-2025.
Ting, J. P., and Trowsdale, J. (2002). Genetic control of MHC class II expression. Cell 109 Suppl, S21-33.
Vantourout, P., and Hayday, A. (2013). Six-of-the-best: unique contributions of gammadelta T cells to immunology. Nat Rev Immunol 13, 88-100.
Wang, D., Quan, Y., Yan, Q., Morales, J. E., and Wetsel, R. A. (2015). Targeted Disruption of the beta2-Microglobulin Gene Minimizes the Immunogenicity of Human Embryonic Stem Cells. Stem Cells Transl Med 4, 1234-1245.
Willingham, S. B., Volkmer, J. P., Gentles, A. J., Sahoo, D., Dalerba, P., Mitra, S. S., Wang, J., Contreras-Trujillo, H., Martin, R., Cohen, J. D., et al. (2012). The CD47-signal regulatory protein alpha (SIRPa) interaction is a therapeutic target for human solid tumors. Proc Natl Acad Sci USA 109, 6662-6667.
Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., et al. (2007). Induced pluripotent stem cell lines derived from human somatic cells. Science 318, 1917-1920.
Zhao, H., Wang, J., Kong, X., Li, E., Liu, Y., Du, X., Kang, Z., Tang, Y., Kuang, Y., Yang, Z., et al. (2016). CD47 Promotes Tumor Invasion and Metastasis in Non-small Cell Lung Cancer. Sci Rep 6, 29719.

1.-56. (canceled)

57. A hypoimmunogenic stem cell comprising three exogenous genes encoding PD-L1, HLA-G, and CD47, respectively, wherein the three exogenous genes are inserted into a safe harbor locus of at least one allele of the hypoimmunogenic stem cell.

58. The hypoimmunogenic stem cell of claim 57, further comprising reduced expression of HLA-A, HLA-B, HLA-C, and CIITA.

59. The hypoimmunogenic stem cell of claim 58, wherein the hypoimmunogenic stem cell does not express HLA-A, HLA-B, HLA-C, and CIITA.

60. The hypoimmunogenic stem cell of claim 57, wherein the hypoimmunogenic stem cell retains differentiation potential.

61. The hypoimmunogenic stem cell of claim 57, wherein the hypoimmunogenic stem cell elicits a reduced adaptive immune response and a reduced innate immune response in a subject into which the hypoimmunogenic stem cell is transplanted as compared to responses elicited by a wild type stem cell.

62. The hypoimmunogenic stem cell of claim 57, wherein the hypoimmunogenic stem cell elicits reduced immune responses from T cells, NK cells, and/or macrophages in a subject into which the hypoimmunogenic stem cell is transplanted as compared to responses elicited by a wild type stem cell.

63. The hypoimmunogenic stem cell of claim 62, wherein the T cells are CD8+ T cells.

64. The hypoimmunogenic stem cell of claim 57, wherein the safe harbor locus is an AAVS1 locus.

65. The hypoimmunogenic stem cell of claim 57, wherein the safe harbor locus is a HPRT locus.

66. A nucleic acid molecule comprising:
i) an expression cassette comprising:
a) a first nucleic acid sequence encoding PD-L1,
b) a second nucleic acid sequence encoding HLA-G, and
c) a third nucleic acid sequence encoding CD47,
ii) a promoter that induces overexpression of PD-L1, HLA-G, and CD47, and
iii) a first arm sequence and a second arm sequence, wherein the first arm sequence is located upstream of the expression cassette and the promoter and the second arm sequence is located downstream of the expression cassette and the promoter, and wherein the arm sequences are homologous to a safe harbor locus of a target stem cell.

67. The nucleic acid molecule of claim 66, wherein the safe harbor locus is an AAVS1 locus.

68. The nucleic acid molecule of claim 66, wherein the safe harbor locus is a HPRT locus.

69. The nucleic acid molecule of claim 66, wherein the promoter is a CAGGS promoter.

70. A hypoimmunogenic stem cell comprising two exogenous genes encoding PD-L1 and CD47, respectively, wherein the two exogenous genes are inserted into a safe harbor locus of at least one allele of the hypoimmunogenic stem cell.

71. The hypoimmunogenic stem cell of claim 70, further comprising reduced expression of HLA-A, HLA-B, HLA-C, and CIITA.

72. The hypoimmunogenic stem cell of claim 71, wherein the hypoimmunogenic stem cell comprises no expression of HLA-A, HLA-B, HLA-C, and CIITA.

73. The hypoimmunogenic stem cell of claim 70, wherein the hypoimmunogenic stem cell retains differentiation potential.

74. The hypoimmunogenic stem cell of claim 70, wherein the hypoimmunogenic stem cell elicits reduced immune responses from T cells and/or macrophages in a subject into which the hypoimmunogenic stem cell is transplanted as compared to immune responses elicited by a wild type stem cell.

75. The hypoimmunogenic stem cell of claim 74, wherein the T cells are CD8+ T cells.

76. The hypoimmunogenic stem cell of claim 70, wherein the safe harbor locus is an AAVS1 locus.

77. The hypoimmunogenic stem cell of claim 70, wherein the safe harbor locus is a HPRT locus.

78. A nucleic acid molecule comprising:
i) an expression cassette comprising:
a) a first nucleic acid sequence encoding PD-L1, and
b) a second nucleic acid sequence encoding CD47,
ii) a promoter that induces overexpression of PD-L1 and CD47, and
iii) a first arm sequence and a second arm sequence, wherein the first arm sequence is located upstream of the expression cassette and the promoter and the second arm sequence is located downstream of the expression cassette and the promoter, and wherein the arm sequences homologous to a safe harbor locus of a target stem cell.

79. The nucleic acid molecule of claim 78, wherein the safe harbor locus is an AAVS1 locus.

80. The nucleic acid molecule of claim 78, wherein the safe harbor locus is a HPRT locus.

81. The nucleic acid molecule of claim 78, wherein the promoter is a CAGGS promoter.


SEQUENCE LISTING (79 sequences)

SEQ ID NO: 1 - DNA, 19 bp, Artificial
  CRISPR gRNA
gccgcctcccacttgcgct

SEQ ID NO: 2 - DNA, 20 bp, Artificial
  CRISPR gRNA
cacatgcagcccacgagccg

SEQ ID NO: 3 - DNA, 20 bp, Artificial
  CRISPR gRNA
atccctaaatatggtgtccc

SEQ ID NO: 4 - DNA, 20 bp, Artificial
  CRISPR gRNA
tccctaaatatggtgtccct

SEQ ID NO: 5 - DNA, 20 bp, Artificial
  CRISPR gRNA
gtgatccgggtatgggcagt

SEQ ID NO: 6 - DNA, 20 bp, Artificial
  CRISPR gRNA
tgatccgggtatgggcagtg

SEQ ID NO: 7 - DNA, 18 bp, Artificial
  CRISPR gRNA
tccatctggtcatagaag

SEQ ID NO: 8 - DNA, 20 bp, Artificial
  CRISPR gRNA
ggggccactagggacaggat

SEQ ID NO: 9 - DNA, 22 bp, Artificial
  PCR primer
cactcagagcaaaggtcagatg

SEQ ID NO: 10 - DNA, 22 bp, Artificial
  PCR primer
agacttgaatccataagcccaa

SEQ ID NO: 11 - DNA, 22 bp, Artificial
  PCR primer
gacaagtctcggagatggtttt

SEQ ID NO: 12 - DNA, 22 bp, Artificial
  PCR primer
agacttgaatccataagcccaa

SEQ ID NO: 13 - DNA, 22 bp, Artificial
  PCR primer
cactcagagcaaaggtcagatg

SEQ ID NO: 14 - DNA, 22 bp, Artificial
  PCR primer
tttgttgtcagccagacatagg

SEQ ID NO: 15 - DNA, 22 bp, Artificial
  PCR primer
ctggttatctccccattctctg

SEQ ID NO: 16 - DNA, 21 bp, Artificial
  PCR primer
aagcattcactcctgaccctg

SEQ ID NO: 17 - DNA, 22 bp, Artificial
  PCR primer
gtcttccctcccaggcagctca

SEQ ID NO: 18 - DNA, 21 bp, Artificial
  PCR primer
tgaggggtgggggataccgga

SEQ ID NO: 19 - DNA, 20 bp, Artificial
  PCR primer
tcgacctactctcttccgca

SEQ ID NO: 20 - DNA, 20 bp, Artificial
  PCR primer
tagggggcgtacttggcata

SEQ ID NO: 21 - DNA, 20 bp, Artificial
  PCR primer
ccgttctcctgtggattcgg

SEQ ID NO: 22 - DNA, 20 bp, Artificial
  PCR primer
tctctggctccatcgtaagc

SEQ ID NO: 23 - DNA, 20 bp, Artificial
  PCR primer
gtcgcttcagtcaggacaca

SEQ ID NO: 24 - DNA, 20 bp, Artificial
  PCR primer
gaaggtgctgtttggcacag

SEQ ID NO: 25 - DNA, 21 bp, Artificial
  PCR primer
ccttcaacttggacactcggg

SEQ ID NO: 26 - DNA, 18 bp, Artificial
  PCR primer
ccacgggccaactactcc

SEQ ID NO: 27 - DNA, 23 bp, Artificial
  PCR primer
ttacccagttcaatactgagcca

SEQ ID NO: 28 - DNA, 22 bp, Artificial
  PCR primer
aggggtaagctgcaaacttctt

SEQ ID NO: 29 - DNA, 20 bp, Artificial
  PCR primer
gacctccacagggactaggt

SEQ ID NO: 30 - DNA, 20 bp, Artificial
  PCR primer
tttggagttggtgctccctc

SEQ ID NO: 31 - DNA, 20 bp, Artificial
  PCR primer
gccctcatcgagtggtcaaa

SEQ ID NO: 32 - DNA, 20 bp, Artificial
  PCR primer
ccgctagacctgctgctatg

SEQ ID NO: 33 - DNA, 20 bp, Artificial
  PCR primer
ctgggtgtcaatgatggcgt

SEQ ID NO: 34 - DNA, 20 bp, Artificial
  PCR primer
gccacatctaaaggcagtcg

SEQ ID NO: 35 - DNA, 20 bp, Artificial
  PCR primer
gtttgtgggtgctggtcaac

SEQ ID NO: 36 - DNA, 20 bp, Artificial
  PCR primer
ctaggcaacagtgacagggg

SEQ ID NO: 37 - DNA, 20 bp, Artificial
  PCR primer
ggaccatcatgtcggagacc

SEQ ID NO: 38 - DNA, 20 bp, Artificial
  PCR primer
gacctgggagtcacacgaac

SEQ ID NO: 39 - DNA, 20 bp, Artificial
  PCR primer
ctgggtgtcaatgatggcgt

SEQ ID NO: 40 - DNA, 20 bp, Artificial
  PCR primer
gccacatctaaaggcagtcg

SEQ ID NO: 41 - DNA, 20 bp, Artificial
  PCR primer
aagtgttcggtctgcgagaa

SEQ ID NO: 42 - DNA, 20 bp, Artificial
  PCR primer
gctctgcttggtacggactg

SEQ ID NO: 43 - DNA, 20 bp, Artificial
  PCR primer
aggtgatgtatggctgcgac

SEQ ID NO: 44 - DNA, 20 bp, Artificial
  PCR primer
tccttcccgttctccaggta

SEQ ID NO: 45 - DNA, 20 bp, Artificial
  PCR primer
ggtatgaacagcacgccaac

SEQ ID NO: 46 - DNA, 20 bp, Artificial
  PCR primer
gcgtcttgtgttccctggta

SEQ ID NO: 47 - DNA, 20 bp, Artificial
  PCR primer
accctctacctgggagaacc

SEQ ID NO: 48 - DNA, 20 bp, Artificial
  PCR primer
aggctctcctttgttcagcc

SEQ ID NO: 49 - DNA, 20 bp, Artificial
  PCR primer
cctagccacgtgtgactcaa

SEQ ID NO: 50 - DNA, 20 bp, Artificial
  PCR primer
tgccgtcccagtaaccaatc

SEQ ID NO: 51 - DNA, 20 bp, Artificial
  PCR primer
cgagggagggcaaattgagt

SEQ ID NO: 52 - DNA, 20 bp, Artificial
  PCR primer
gaagaagggacaaggggtgg

SEQ ID NO: 53 - DNA, 22 bp, Artificial
  PCR primer
gagctttgatggcagagtgtta

SEQ ID NO: 54 - DNA, 22 bp, Artificial
  PCR primer
ctgggagcatgcatttgtgaga

SEQ ID NO: 55 - DNA, 20 bp, Artificial
  PCR primer
ctgttggcaggtgaacctct

SEQ ID NO: 56 - DNA, 20 bp, Artificial
  PCR primer
ctgggaaggcttgtctcctg

SEQ ID NO: 57 - DNA, 20 bp, Artificial
  PCR primer
agagctcaaggtcccaagtg

SEQ ID NO: 58 - DNA, 20 bp, Artificial
  PCR primer
gggtaactcactcggaggtc

SEQ ID NO: 59 - DNA, 21 bp, Artificial
  PCR primer
tggatttccagaaccccatcc

SEQ ID NO: 60 - DNA, 20 bp, Artificial
  PCR primer
gggaactctccctctggtct

SEQ ID NO: 61 - DNA, 20 bp, Artificial
  PCR primer
gagcttctgcattggtccct

SEQ ID NO: 62 - DNA, 20 bp, Artificial
  PCR primer
tctgatcgatctgccctcct

SEQ ID NO: 63 - DNA, 20 bp, Artificial
  SYBR-based qPCR primers
aaatgcgtttctcgttgctt

SEQ ID NO: 64 - DNA, 20 bp, Artificial
  SYBR-based qPCR primers
gccacaggccaatagtttgt

SEQ ID NO: 65 - DNA, 18 bp, Artificial
  SYBR-based qPCR primers
ctctgcctcctccacgaa

SEQ ID NO: 66 - DNA, 20 bp, Artificial
  SYBR-based qPCR primers
cagaatccagacctgcacaa

SEQ ID NO: 67 - DNA, 20 bp, Artificial
  SYBR-based qPCR primers
aattggtccagccttggaat

SEQ ID NO: 68 - DNA, 18 bp, Artificial
  SYBR-based qPCR primers
cgttgctcacagaccaca

SEQ ID NO: 69 - DNA, 20 bp, Artificial
  SYBR-based qPCR primers
tgatcggaaatgacactgga

SEQ ID NO: 70 - DNA, 20 bp, Artificial
  SYBR-based qPCR primers
cacgactccatgttggtcac

SEQ ID NO: 71 - DNA, 30 bp, Artificial
  SYBR-based qPCR primers
caggtggcggacgtgtgaaaattgagagtg

SEQ ID NO: 72 - DNA, 26 bp, Artificial
  SYBR-based qPCR primers
cacgctggatctgcctggggactgtg

SEQ ID NO: 73 - DNA, 20 bp, Artificial
  SYBR-based qPCR primers
gtccatctttgcttgggaaa

SEQ ID NO: 74 - DNA, 20 bp, Artificial
  SYBR-based qPCR primers
tagccaggttgcgaagaact

SEQ ID NO: 75 - DNA, 20 bp, Artificial
  CRISPR sgRNAs used to target the first coding exon of CD47 in HUES8 cells
ggtcctgcctgtaacggcgg

SEQ ID NO: 76 - DNA, 20 bp, Artificial
  CRISPR sgRNAs used to target the first coding exon of CD47 in HUES8 cells
ggaccgccgccgcgcgtcac

SEQ ID NO: 77 - DNA, 20 bp, Artificial
  CRISPR sgRNAs used to target the first coding exon of CD47 in HUES8 cells
gcagcaacagcgccgctacc

SEQ ID NO: 78 - DNA, 20 bp, Artificial
  CRISPR sgRNAs used to target the first coding exon of CD47 in HUES8 cells
gttcgcccccgcgggcgtgt

SEQ ID NO: 79 - DNA, 20 bp, Artificial
  sgRNA used to generate Beta-2-Microglobulin (B2M)-deficient HUES8 cell line
gctactctctctttctggcc